SEQUENCE LISTING (22 sequences)

SEQ ID NO: 1 - RNA, 22 nt, Homo sapiens
ucucccaacccuuguaccagug

SEQ ID NO: 2 - RNA, 22 nt, Homo sapiens
uagcagcacaucaugguuuaca

SEQ ID NO: 3 - RNA, 23 nt, Homo sapiens
cccaguguucagacuaccuguuc

SEQ ID NO: 4 - RNA, 21 nt, Homo sapiens
gugcauuguaguugcauugca

SEQ ID NO: 5 - RNA, 23 nt, Homo sapiens
ugaggggcagagagcgagacuuu

SEQ ID NO: 6 - RNA, 22 nt, Homo sapiens
cagcagcaauucauguuuugaa

SEQ ID NO: 7 - RNA, 22 nt, Homo sapiens
agagguaguagguugcauaguu

SEQ ID NO: 8 - RNA, 23 nt, Homo sapiens
agcagcauuguacagggcuauga

SEQ ID NO: 9 - RNA, 21 nt, Homo sapiens
aucacauugccagggauuacc

SEQ ID NO: 10 - RNA, 22 nt, Homo sapiens
uguaaacauccccgacuggaag

SEQ ID NO: 11 - RNA, 23 nt, Homo sapiens
aaugacacgaucacucccguuga

SEQ ID NO: 12 - RNA, 21 nt, Homo sapiens
agcuacaucuggcuacugggu

SEQ ID NO: 13 - RNA, 21 nt, Homo sapiens
aucacauugccagggauuucc

SEQ ID NO: 14 - RNA, 22 nt, Homo sapiens
uucaaguaauccaggauaggcu

SEQ ID NO: 15 - RNA, 23 nt, Homo sapiens
ugagcgccucgacgacagagccg

SEQ ID NO: 16 - RNA, 23 nt, Homo sapiens
agcuacauugucugcuggguuuc

SEQ ID NO: 17 - RNA, 23 nt, Homo sapiens
uguaaacauccuacacucucagc

SEQ ID NO: 18 - RNA, 23 nt, Homo sapiens
aacauucaacgcugucggugagu

SEQ ID NO: 19 - RNA, 23 nt, Homo sapiens
uguaguguuuccuacuuuaugga

SEQ ID NO: 20 - RNA, 22 nt, Homo sapiens
aaaccguuaccauuacugaguu

SEQ ID NO: 21 - RNA, 21 nt, Homo sapiens
uucacaguggcuaaguucugc

SEQ ID NO: 22 - RNA, 21 nt, Homo sapiens
aucacauugccagggauuucc

(19) United States
(12) Patent Application Publication
(10) Pub. No.: US 2015/0344961 A1
(43) Pub. Date: Dec. 3, 2015

(54) Sera Based miRNAs as Non-Invasive Biomarkers in Melanoma

(71) Applicant: NEW YORK UNIVERSITY, New York, NY (US)

(72) Inventors: Iman Osman, Jersey City, NJ (US); Erica Friedman, New York, NY (US); Eleazar Vega-Saenz De Meira, New York, NY (US); Yongzhao Shao, Forest Hills, NY (US); Shulian Shang, New York, NY (US); Eva Hernando, New York, NY (US)

(21) Appl. No.: 14/427,352

(22) PCT Filed: Sep. 11, 2013
(86) PCT No.: PCT/US2013/059128

§ 371 Date: Mar. 11, 2015

Related U.S. Application Data
(60) Provisional application No. 61/699,294, filed on Sep. 11, 2012.

(51) Int. Cl. C12Q 1/68

(52) U.S. Cl. CPC C12Q 1/6886; C12Q 2600/178; C12Q 2600/158; C12Q 2600/118

(57) ABSTRACT

The present invention relates to serum marker microRNAs (miRNAs) which are associated with cancer, particularly melanoma, and to the assessment thereof in the prognosis, treatment and management of cancer. Embodiments include methods, compositions, kits and isolated nucleic acids. The present invention is directed to methods and compositions for prognosing melanoma and monitoring for recurrence by monitoring serum miRNAs, and the use of miRNAs and antagonists thereof, particularly antagomirs, for predicting and assessing risk and/or likelihood of recurrence in a melanoma patient. The present invention relates to biomarkers for melanoma, particularly serum markers and sets thereof which are relevant and significant as prognostic indicators of melanoma disease and patient risk for recurrence.

### GOVERNMENTAL SUPPORT

This invention was made with government support under Grant 1UL1RR029893 from the National Center for Research Resources, National Institutes of Health and the NYU Cancer Institute Cancer Center Support Grant 5P30CA16087-27. The government has certain rights in the invention.

### FIELD OF THE INVENTION

The present invention relates to biomarkers for melanoma, particularly serum markers and sets thereof which are relevant and significant as prognostic indicators of melanoma disease and patient risk for recurrence and as markers of melanoma relapse. The invention relates to serum expression of microRNAs (miRNAs) and their assessment for prognosis of melanoma.

### BACKGROUND OF THE INVENTION

Melanoma remains a highly morbid disease in the United States and its incidence has continued to rise sharply over the past few decades (Altekruse S F et al (eds). SEER Cancer Statistics Review, 1975-2007 (Bethesda, Md.: National Cancer Institute) updated based on November 2009 SEER data submission, posted to the SEER web site, 2010. Jan. 31, 2011; seer.cancer.gov/csr/1975_{—}2007). In addition, the toll of melanoma in terms of “life-years lost” is the highest of all solid tumors in the United States (Tsao H et al (2004) N Engl J Med 351:998-1012). In the absence of curative therapy for patients with metastatic melanoma, the identification of patients at high risk for recurrence at the time of primary diagnosis is critical for informed management decisions.

Conventional approaches to treating cancer, including melanoma, and to determining prognosis, detecting relapse, predicting and assessing cancer and cancer cell responses to specific treatment regimens rely on properly classifying the type of tumor present. Proper classification, in turn, relies primarily on clinical features, such as tumor depth and tumor cell morphology. The current standards of care for both determining the prognosis of melanoma patients with localized disease and guiding postoperative follow-up have limitations. Using prognostic factors, such as Breslow depth, primary ulceration, mitotic rate and lymph node involvement, the current American Joint Committee on Cancer (AJCC) staging system is able to classify melanoma patients into stages that provide first line stratification in recurrence risk (Balch C M et al (2001) J Clin Oncol 19:3635-3648). However, the current system only partly explains the variability in the prognosis of melanoma and there remains unexplained heterogeneity within each stage. Currently utilized markers, such as lactate dehydrogenase and S-100 beta, only showing prognostic significance in advanced disease (Balch C M et al (2001) J Clin Oncol 19:3635-3648; Deichmann M et al (1999) J Oncol 17:1891-1896; Tarhini A A et al (2009) J Clin Oncol 27:38-44; Kruijff, S et al (2009) Ann Surg Oncol 16:3455-3462; Hauschild A et al (1999) Oncology 56:338-344). Additionally, despite the benefit of early detection of loco-regional and distant metastasis amenable to curative resection (Garbe, C (2002) Recent Res Cancer Res 160:205-215; Leiter, U (2010) Melanoma Res 20(3):240-246), there is no consensus on selection and timing of imaging studies and laboratory tests for use in follow-up and existing guidelines are not consistently applied (Grange, F et al (2008) Arch Dermatol 144(5):629-636). This is in part due to the limited sensitivity and specificity of available imaging modalities and blood tests, coupled with considerable economic cost (Hofmann, U et al (2002) Br J Cancer 87(2):151-157).

Increasing evidence implicates aberrant expression of microRNAs (miRNAs) in many human malignancies, and suggests that they may indeed act as either/both tumor suppressors or as oncogenes, and can have effects in numerous cancers which may have a common pathway, or in specific cancers which have particular miRNA initiators or modulators (Visone R and Croce C M (2009) Am J Pathology 174(4):1131-1138; Garzon R et al (2009) Ann Rev Med 60:167-179; Lui W-O et al (2007) Cancer Res 67(13):6031-6043).

MicroRNAs (miRNAs) are small, non-coding RNAs that are ubiquitous regulators of biological processes involved in normal development, in differentiation and in diseases, including cancer. They act by regulating gene expression at the transcriptional and translational levels (Bartel et al (2004) Cell 116:281-297). miRNAs were initially discovered by analysis of mutations causing developmental defects in Caenorhabditis elegans (Lee R. C. et al (1993) Cell, 75, 843-854) and altered miRNA expression has been further demonstrated in human cancer (Calin G. A. et al (2004) PNAS USA 101: 11755-11760; Hayashita Y et al (2005) Cancer Res 65:9628-9632; Johnson S. M. et al (2005) Cell 120:635-647; Lu J et al (2005) Nature 435:834-838; Venturini L et al (2007) Blood 109: 4399-4405). MicroRNAs (miRNA) regulate gene expression in a sequence specific manner by hybridization and recruitment of multi-protein complexes to complementary messenger RNA (mRNA) target sequences. miRNA function can transiently be antagonized by antagomirs—chemically modified oligonucleotides complementary to individual miRNAs.

A single miRNA can target hundreds of messenger RNAs and thereby modulate protein output from their respective genes (Bartel D P (2009) Cell 136:215-233). Therefore a single or specific set of miRNAs may control discrete physiological processes by regulating the production of a few critical proteins that coordinate single or interrelated cellular events (e.g., cell proliferation) (Baltimore D et al (2008) Nat Immunol 9:839-845; Bartel D P (2009) Cell 136:215-233).

Ideal prognostic biomarkers are sensitive, specific, reproducible and measurable in easily accessible samples. Due to the routine collection and facility of obtaining blood samples at multiple time points, blood-based biomarkers are a logical and cost effective source in the search for non-invasive markers. MicroRNAs are present in human plasma and serum in stable form and resistant to RNase digestion, harsh conditions, extended storage, and multiple freeze-thaw cycles (Mitchell P S et al (2008) Proc Natl Acad Sci USA 105:10513-10518; Chen X et al (2008) Cell Res 18:997-1006), making them promising cancer biomarkers.

Although numerous miRNAs are known and have been identified (known miRNAs are accessible by name with sequence information and characteristics via public database(s) including the miRBase database, mirbase.org; Griffiths-Jones S (2003) Methods Mol Biol 342:129-138), their specific roles in initiation and/or progression of disease(s) and their particular value as targets for therapies or as modulators of disease, including cancer remain largely ill-defined. Studies assessing miRNAs in plasma and serum as biomarkers in a variety of cancers have largely focused on distinguishing cancer patients from control subjects (Cortez M A et al (2011) Nat Rev Clin Oncol 8(8):467-477; Cortez M A et al (2009) Expert Opin Biol Ther 9:703-711). Little focus has been placed on evaluating the prognostic potential of circulating miRNAs (Lawrie C H et al (2008) Br J Haematol 141:672-675; Cheng H et al (2011) PLoSONE 6:e17745; Hu Z et al (2010) J Clin Oncol 28:1721-1726; Boeri M et al (2011) Proc Natl Acad Sci USA 108:3713-3718; Ali S et al (2010) Am J Transl Res 3:28-47; Brase J C et al (20100) Int J Cancer 128:608-616). To date, no study has specifically examined the prognostic utility of combining sera miRNAs and clinical characteristics, at the time of primary diagnosis, in identifying melanoma patients at high risk of disease recurrence.

Therefore, it should be apparent that there still exists a need for specific elucidation of the relevance of individual or collective miRNAs in cancers, including in specific cancers, for prognosis, and management of disease. The present invention demonstrates the use and application of serum miRNAs for melanoma in monitoring for disease recurrence and surveillance and for prognosis and the additional prognostic value of incorporating a set of serum miRNAs into a model with clinicopathological covariates in evaluating and predicting recurrence of primary melanoma patients.

The citation of references herein shall not be construed as an admission that such is prior art to the present invention.

### SUMMARY OF THE INVENTION

In its broadest aspect, the present invention extends to methods, markers and compositions for predicting prognosis of melanoma cancer patients and monitoring for disease progression. Specific miRNAs are provided as indicators of prognosis, including risk of recurrence and recurrence of cancer, particularly melanoma, based on their expression in serum of patients. Thus, selective sets of miRNAs provide serum markers which, particularly in combination with clinical covariates or indicators, provide information on the prognosis of melanoma patients and the risk or likelihood or recurrence in a melanoma patient. The miRNAs provided are identified, characterized and assessed from patient serum, providing a particularly applicable prognostic and surveillance test, assay, or method utilizing a non-invasive and readily obtainable patient sample.

The data presented herein demonstrate that miRNAs, particularly one or more, particularly at least two, particularly at least three, particularly at least four, particularly at least five, particularly at least six, particularly at least seven, including at least 8, particularly four or more, particularly five or more, particularly six or more, particularly seven or more, or any relevant combination of at least two, at least three, at least four, at least five, at least six, at least seven, at least eight, at least nine of a group of serum marker miRNAs selected from the miRNAs miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p are useful in the monitoring of cancer, and particularly the prognosis of cancer in a patient, particularly melanoma. In a particular aspect the patient is a human cancer patient, a human with melanoma, and the miRs are Homo sapien miRs or has-MiRs.

In accordance with the invention, information on the expression or amounts of a set of miRNAs, selected from among the miRs provided herein, listed in Table 1, or a miRNA set comprising at least three of miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p, can be used for the assessment and prognosis of melanoma in a subject, for predicting likelihood or risk of recurrence. Those melanoma patients with altered expression of miRs selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p have been found to have a greater risk of recurrence and have a shorter disease free survival time.

In an aspect of the invention, information on the expression or amounts of a set of miRNAs, particularly miRNAs miR-425, miR-150, miR-15b, and miR-23b, can be used for the assessment and prognosis of melanoma in a subject, for predicting likelihood or risk of recurrence. In a particular aspect, information on the expression or amounts of miRNAs miR-425/mir-425-5p, miR-150/miR-150-5p, miR-15b/miR-15b-5p, and miR-23b/miR-23b-3p can be used for the assessment and prognosis of melanoma in a subject, for predicting likelihood or risk of recurrence. In a particular such aspect the subject is a stage I patient.

In an additional aspect of the invention, information on the expression or amounts of a set of miRNAs (marker miRNAs) is compared to information on the expression or amounts of a set of normalization or control miRNAs and then used for assessmemt and prognosis of melanoma in a subject, for predicting likelihood or risk of recurrence. In one such aspect, information on the expression or amounts of a set of miRNAs (marker miRNAs) is compared to information on the expression or amounts of a set of normalization or control miRNAs, wherein the normalization or control miRNAs are or are selected from miR-142/miR-142-3p, miR451/miR451a, mir-30c/miR-30c-5p, miR-181a/miR-181a-5p, miR-27b/miR-27b-3p and miR-23a/miR-23a-3p. In an aspect, information on the expression or amounts of a set of miRNAs (marker miRNAs) is compared to information on the expression or amounts of a set of normalization or control miRNAs, wherein the normalization miRNAs are mir-30c/miR-30c-5p and miR-181a/miR-181a-5p. In a further aspect, information on the expression or amounts of a set of miRNAs (marker miRNAs) miR-425/miR-425-5p, miR150/miR-150-5p, miR23b/miR-23b-3p and miR15b/miR-15b-5p is compared to information on the expression or amounts of a set of normalization or control miRNAs, wherein an a particular aspect the normalization miRNAs are mir-30c/miR-30c-5p and miR-181a/miR-181a-5p.

The prognostic and medical utility of the present invention extends to the use of the present serum microRNA markers in assays to evaluate and/or monitor cancer, malignancy, risk and/or likelihood or recurrence, and/or recurrence in a mammal, particularly a human patient, particularly with regard to melanoma cancer. Thus, methods for monitoring melanoma progression and/or evaluating or predicting response to treatment and/or determining risk or likelihood of recurrence are provided wherein the expression or activity of one or more miRNA selected from the microRNAs provided herein is assessed. In one such aspect, the activity of at least two, at least three, at least four, at least five, at least six, at least seven miRNAs associated with cancer, selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p is determined and assessed, so as to monitor or evaluate melanoma cancer, recurrence of cancer, or response to cancer treatment, such as at the molecular level. For instance, altered expression and/or activity of at least two, at least three, at least four, at least five, at least six, at least seven miRNAs associated with cancer, selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p may be prognostic and may be associated with recurrence or an increased risk or likelihood of recurrence in a subject at risk of, with, or having been diagnosed with melanoma.

The present invention relates to methods of monitoring or predicting recurrence risk in subjects having melanoma via methods for determining the expression or amount of a set of miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p. The invention provides serum biomarkers for determining melanoma prognosis and predicting recurrence in a subject diagnosed with or having primary melanoma cancer, comprising or selected from specific miRNAs. The serum biomarkers comprise a set of miRNAs, particularly two or more of, three or more of, four or more of, five or more of, six or more of, seven or more of, at least four of, at least five of, at least six of, at least seven of, or all of miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p. The number of miRNA biomarkers may vary and will be sufficient in number to provide a sensitive and specific prognostic value for determining recurrence or risk of recurrence in a patient. The miRNA biomarkers may comprise two or more of, three or more of, four or more of, five or more of, six or more of, seven or more of, at least four of, at least five of, at least six of, at least seven of, or all of miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p, and may or may not further comprise additional miRNAs with prognostic value.

In preferred aspects of the methods of the invention, methods and/or kits are provided for monitoring or predicting recurrence risk in subjects having melanoma by determining the expression or amount of a set of miRNAs selected from:
(a) miRNAs miR-150, miR-15b, miR-199-5p, miR-33a, miR-423-5p, miR-424, and miR-let-7d;
(b) miRNAs miR-150, miR-15b, miR-199-5p, miR-33a, and miR-424;
(c) miRNAs miR-423-5p, miR-424, and miR-199a-5p;
(d) miRNAs miR-103, mir-221, miR-222, and miR-423-5p; and
(e) miRNAs miR-425, miR-150, miR-23b and miR-15b.

In preferred aspect of the methods of the invention, methods and/or kits are provided for monitoring or predicting recurrence risk in subjects having melanoma by determining the expression or amount of a set of miRNAs:
(a) miRNAs miR-425, miR-150, miR-23b and miR-15b; and
(b) miRNAs miR-30c and miR-181a.

The miRNAs may be assessed, quantified and/or identified in serum, thereby facilitating collection from and assessment of a cancer patient, particularly a melanoma patient.

In an aspect of the invention a preferred set of serum miRNAs for analysis in a melanoma patient and with significance in methods, kits, assays to distinguish recurring from non-recurring melanoma patients or for prognosis of recurrence may be a set comprising miRNAs miR-150, miR-15b, miR-199-5p, miR-33a, miR-423-5p, miR-424, and miR-let-7d. In a further aspect, a preferred set of serum miRNAs for analysis in a melanoma patient and with significance in methods, kits, assays to distinguish recurring from non-recurring melanoma patients or for prognosis of recurrence may be a set comprising miRNAs miR-103, miR-15b, miR-23b, miR-30d, miR-423-5p and miR-425. In an additional aspect, a preferred set of serum miRNAs for analysis in a melanoma patient and with significance in methods, kits, assays to distinguish recurring from non-recurring melanoma patients or for prognosis of recurrence may be a set comprising miRNAs miR-222, miR-23a, miR-26a, miR-339-3p and miR-423.5p. In an aspect of the invention a preferred set of serum miRNAs for analysis in a melanoma patient and with significance in methods, kits, assays to distinguish recurring from non-recurring melanoma patients or for prognosis of recurrence may be a set comprising or having miRNAs miR-425, miR-150, miR-23b and miR-15b.

The invention provides serum miRNAs which may be selected in a set of serum miRNAs for analysis in a melanoma patient with, for example, Stage I, II or III melanoma disease and with significance in distinguishing or predicting recurring from non-recurring melanoma, or for predicting risk of recurrence, even in early/earlier stage of disease. In an aspect of the invention a set of serum miRNAs may be provided for analysis in a melanoma patient with Stage II melanoma and with significance in methods, kits, assays to distinguish recurring from non-recurring Stage II melanoma patients or for prognosis of recurrence in a patient with diagnosed Stage II melanoma. In an aspect, a set of serum miRNAs may be a set comprising miRNAs miR-30d, miR-199-5p, miR-222, miR-423.5p and miR-424.

In an aspect of the invention a set of serum miRNAs may be provided for analysis in a melanoma patient with Stage I melanoma and with significance in methods, kits, assays to distinguish recurring from non-recurring Stage I melanoma patients or for prognosis of recurrence in a patient with diagnosed Stage I melanoma. In an aspect, a set of serum miRNAs may be a set comprising miRNAs miR-425, miR-150, miR-23b and miR-15b. In an aspect, a set of serum miRNAs may be a set comprising marker miRNAs miR-425, miR-150, miR-23b and miR-15b and normalization miRNAs miR-30c and miR-181a.

The present invention further includes an assay system which may be prepared in the form of a test kit for the quantitative analysis of the extent of the presence of the miRNAs hereof, or to identify drugs or other agents that may mimic or block their activity. Thus, an assay system may be prepared and is provided in the form of a test kit for the analysis or determination of the presence, including the extent of the presence of the microRNAs provided herein, particularly as set out in Table 1, particularly a combination of microRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p, and may or may not further comprise additional miRNAs with prognostic and/or diagnostic value, or other clinical parameters or indicators.

In an aspect of the invention, a method is provided for monitoring melanoma cancer or evaluating risk, probability or likelihood of cancer recurrence in a mammal comprising:

(a) obtaining a cellular, blood or serum sample from said mammal;
(b) measuring the expression or activity of at least two miRNAs miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p in said sample; and
(c) optionally comparing the expression or activity of said at least two miRNAs to that in or from a reference sample or a reference or normal value or to at least one miRNAs serving as normalization or control miRNAs;
wherein the expression or activity of at least two of said miRNAs is altered relative to the reference sample or a reference or normal value or a normalization or control miRNA. In one aspect of this method, the expression or activity of three or more miRNAs is measured, compared, and altered. In aspects of this method, the expression or activity of four or more miRNAs, five or more miRNAs, six or more miRNAs, seven or more miRNAs, nine or more miRNAs, ten or more miRNAs, is measured, compared, and altered. In an additional aspect, the melanoma cancer is at any stage including Stage I, II or III or undefined or uncharacterized. The method may involve obtaining one or more sample, particularly one or more serum ample, from a mammal or melanoma patient over the course of treatment and/or post-treatment or over the course of the disease.

In an aspect of the invention, a method is provided for monitoring melanoma cancer or evaluating risk, probability or likelihood of cancer recurrence in a mammal comprising:

(d) obtaining a cellular, blood or serum sample from said mammal;
(e) measuring the expression or activity of miRNAs miR-150, miR15b, miR-23b, and miR-425 in said sample; and
(f) optionally comparing the expression or activity of said at least two miRNAs to that in or from a reference sample or a reference or normal value or to at least one miRNAs serving as normalization or control miRNAs;
wherein the expression or activity of at least two of said miRNAs is altered relative to the reference sample or a reference or normal value or a normalization or control miRNA.

The invention further provides a method for prognosis of melanoma in a subject, which method comprises (a) providing a biological sample from the subject; (b) obtaining an expression profile of at least three nucleic acid sequences selected from the group consisting of SEQ ID NOS: 1-15 and sequences at least about 80% identical thereto from said sample; and (c) comparing said obtained expression profile to a reference expression profile; wherein a differential expression of the nucleic acid in the subject as compared to the reference expression profile provides a prognosis for the subject having melanoma.

The invention further provides a method for prognosis of melanoma in a subject, which method comprises (a) providing a biological sample from the subject; (b) obtaining an expression profile of at least three nucleic acid sequences selected from the group consisting of SEQ ID NOS: 1-15 and sequences at least about 80% identical thereto from said sample; and (c) comparing said obtained expression profile to a reference expression profile or to the reference expression profile of at least two nucleic acid sequences selected from the group consisting of SEQ ID NOS: 17-22 and sequences at least about 80% identical thereto; wherein a differential expression of the nucleic acid in the subject as compared to the reference expression profile provides a prognosis for the subject having melanoma.

The invention provides a method for prognosis of melanoma in a subject, which method comprises (a) providing a biological sample from the subject; (b) obtaining an expression profile of nucleic acid sequences SEQ ID NOS: 1, 2, 9 and 11 and sequences at least about 80% identical thereto from said sample; and (c) comparing said obtained expression profile to a reference expression profile or to the reference expression profile of at least two nucleic acid sequences selected from the group consisting of SEQ ID NOS: 17-21 and sequences at least about 80% identical thereto; wherein a differential expression of the nucleic acid in the subject as compared to the reference expression profile provides a prognosis for the subject having melanoma. In an aspect of the method, in part (c) the nucleic acid sequences are SEQ ID NOS: 17 and 18.

The biological sample can be selected from the group consisting of bodily fluid, a cellular sample, a cell line and a tissue sample. The bodily fluid may be blood, serum or urine. In an aspect of the invention, the biological sample is serum. The tissue can be selected from fresh, frozen, fixed, wax-embedded or formalin fixed paraffin-embedded (FFPE) tissue.

A kit for prognosis of a subject with melanoma is also provided. In a specific embodiment, the kit comprises at least one probe comprising a nucleic acid sequence that is complementary to one or more sequence(s) selected from the miRNA sequences provided herein, the miRNAs set out in Table 1, SEQ ID NO: 1-15; or to a sequence at least about 80% identical thereto.

In a particular embodiment, the kit comprises probes comprising nucleic acid sequences complementary to the miRNA sequences set out in SEQ ID NOS: 1, 2, 9 and 11; or to a sequence at least about 80% identical thereto. The kit may further comprising normalization or control probes comprising nucleic acid sequences complementary to at least two miRNA sequences set out in SEQ ID NOS: 17-22; or to a sequence at least about 80% identical thereto, particularly comprising nucleic acid sequences complementary to miRNA sequences set out in SEQ ID NOS: 17 and 18.

In an aspect of the above methods, the expression or activity of two or more miRNAs is measured, compared, and determined to be altered individually or collectively. In one aspect of the above methods, the expression or activity of three or more miRNAs, four or more miRNAs, five or more miRNAs, six or more miRNAs, seven or more miRNAs, at least five miRNAs, at least six miRNAS, at least seven miRNAs is measured, compared, and determined to be altered individually and/or collectively. In a further particular aspect, the cancer is skin cancer. In a further particular aspect, the cancer is melanoma.

In a further embodiment, the invention includes an antisense oligonucleotide or an antagomir comprising a sequence substantially complementary to at least one of the miRNA sequences provided herein, provided in Table 1, selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p. The antagonist or antagomir may be substantially complementary to the miRNA target sequences set out in Table 1. In particular, antagomirs, antagonists or oligonucleotides of the invention include oligonucleotides comprising a sequence substantially complementary to nucleotides selected from the group of SEQ IDs 1-15 as set out in Table 1, or a subset of nucleotides thereof sufficient to alter the expression or activity of one or more of said miRNA sequences SEQ ID NOs 1-15.

In an aspect of the composition of the invention, the one or more antagomir or oligonucleotide comprises at least one modified nucleotide. In a particular aspect, the antagomirs, nucleic acids and oligonucleotides of the present invention may be modified, either by manipulation of the chemical backbone of the nucleic acids or by covalent or non-covalent attachment of other moieties. In each or any case, such manipulation or attachment may serve to modify the stability, cellular, tissue or organ uptake, or otherwise enhance efficacy of the nucleic acids and oligonucleotides. In further aspects of the invention, the antagomirs or oligonucleotides may be covalently linked to other molecules, including but not limited to polypeptides, carbohydrates, lipid or lipid-like moieties, ligands, chemical agents or compounds, which may serve to enhance the uptake, stability or to target the oligonucleotides.

The compositions, probes, antagomirs or oligonucleotides of the present invention may be labeled with a detectable label. In particular aspects, the label may be selected from enzymes, ligands, chemicals which fluoresce and radioactive elements. In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Other objects and advantages will become apparent to those skilled in the art from a review of the following description which proceeds with reference to the following illustrative drawings.

### BRIEF DESCRIPTION OF THE DRAWINGS

FIG. 1. Kaplan-Meier analysis for RFS by recurrence risk defined by the Cox proportional hazards model. Patients defined as high recurrence risk (dashed line) by the Cox proportional hazards model demonstrated significantly reduced RFS compared to patients defined as low recurrence risk (solid line) in both the (A) discovery (p=0.0036 by log rank test) and (B) validation (p=0.009 by log rank test) cohorts.

FIG. 2. Kaplan-Meier analysis for RFS by recurrence risk defined by logistic regression risk model. Patients defined as high recurrence risk (dashed line) by the logistic regression model demonstrated significantly reduced RFS compared to patients defined as low recurrence risk (solid line) in both the (A) discovery (p<0.0001 by log rank test) and (B) validation (p=0.033 by log rank test) cohorts.

FIG. 3. Proof-of-principle logistic regression subgroup analysis of stage II patients. (A) ROC curve for the miRNA containing logistic risk model defined for stage II patients had good classification performance (AUC=0.89). (B) Kaplan-Meier analysis for RFS of high and low recurrence risk groups in stage II patients showed significant separation of RFS curves (p<0.001 by log rank test).

FIG. 4. Longitudinal evaluation of miRNA expression in pre- and post-recurrence serum samples. Difference between miRNA expression levels of miR-103 and miR-221 at primary diagnosis and at recurrence was statistically significant (p=0.012 and p=0.026, respectively). The horizontal axis represents time of blood draw for 17 patients. The vertical axis represents—(Ct) value.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The terms “miRNA(s)”, “miR(s)”, “microRNA(s)”, “miRNA biomarker(s)”, and any variants not specifically listed, may be used herein interchangeably, and as used throughout the present application and claims refer to nucleic acid materials, including ribonucleic acids, RNAs, including single or multiple RNAs, and extends to miRNA(s) including those provided and described herein, those selected from miR-150, miR-15b, miR-199a-5p, miR-33a, miR-423-5p, miR-424, miR-let-7d, miR-103, miR-23b, miR-30d, miR-425, miR-222, miR-23a, miR-26a, miR-339-3p, those having the nucleic acid sequence data described herein and presented in TABLE 1 (SEQ ID NOs: 1-15), and having the profile of activities set forth herein and in the Claims. The miR(s) may be referred to by their sequences, their SEQ ID NOs:, or their miR numbers, with or without suffixes (such as miR-425 or alternatively miR-425p, or miR-23b or miR-23b-3p, etc), including as indicated or provided in TABLE 1. Accordingly, sequences displaying substantially equivalent activity, capability or function and/or altered sequence(s) are likewise contemplated. These modifications may be deliberate, for example, such as modifications obtained through site-directed mutagenesis, or may be accidental, such as those obtained through mutations in hosts or variants thereof. Also, the terms “miRNAs”, “miRs”, “microRNAs” and “miRNA biomarkers” are intended to include within their scope nucleic acids specifically recited herein as well as all substantially homologous analogs and allelic variations.

The terms “oligonucleotides”, “antisense”, “antisense oligonucleotides”, “antagomirs”, “miRNA antagomirs” and any variants not specifically listed, may be used herein interchangeably, and as used throughout the present application and claims refer to nucleic acid material including single or multiple nucleic acids, and extends to those oligonucleotides, probes, or antagomirs complementary to the miRNA nucleic acid sequences described herein, including as presented in TABLE 1 and their complementary sequences and having the profile of activities set forth herein and in the claims, particularly in being capable of altering the expression or activity of one or more miRNAs hereof, particularly inhibiting one or more miRNA provided in Table 1, or one or more RNA selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p. In particular, the oligonucleotides of the present invention may be substantially complementary to nucleic acid sequence specific to miRNA(s) provided or set out herein, to miR(s) selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, as provided in SEQ ID NO: 1-15, or to a portion thereof, as provided for example in TABLE 1. Accordingly, nucleic acids or analogs thereof displaying substantially equivalent activity or function or altered sequence are likewise contemplated. These modifications may be deliberate, for example, such as modifications obtained through site-directed mutagenesis, or may be accidental, such as those obtained as variants or through mutations in hosts that are producers of the nucleic acids or of the antagomirs/oligonucleotides. The term “oligonucleotide” as used herein is defined as a molecule comprised of two or more nucleotides, including ribonucleotides or deoxyribonucleotides, preferably more than three. Its exact size will depend upon many factors which, in turn, depend upon the ultimate function and use of the oligonucleotide.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor or molecule the ligand binds to or associates with in the broadest sense.

As used herein, the term ‘antagonist’ is used to describe a compound that does not provoke a biological response itself upon binding to a receptor, but blocks or dampens agonist-mediated responses.

The term ‘assay’ means any process used to measure a specific property of a compound. A ‘screening assay’ means a process used to characterize or select compounds based upon their activity from a collection of compounds.

The term ‘binding affinity’ is a property that describes how strongly two or more compounds associate with each other in a non-covalent relationship. Binding affinities can be characterized qualitatively, (such as ‘strong’, ‘weak’, ‘high’, or ‘low’) or quantitatively (such as measuring the K_{D}).

The term ‘carrier’ means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term ‘complex’ means the entity created when two or more compounds bind to, contact, or associate with each other.

The term ‘compound’ is used herein in the context of a ‘test compound’ or a ‘drug candidate compound’ described in connection with the assays of the present invention. As such, these compounds comprise organic or inorganic compounds, derived synthetically, recombinantly, or from natural sources.

The term ‘fragment of a polynucleotide’ relates to oligonucleotides that comprise a stretch of contiguous nucleic acid residues that exhibit substantially a similar, but not necessarily identical, activity as the complete sequence. In a particular aspect, ‘fragment’ may refer to a oligonucleotide comprising a nucleic acid sequence of at least 5 nucleic acid residues (preferably, at least 10 nucleic acid residues, at least 15 nucleic acid residues, at least 20 nucleic acid residues, at least 25 nucleic acid residues, at least 40 nucleic acid residues, at least 50 nucleic acid residues, at least 60 nucleic residues, at least 70 nucleic acid residues, at least 80 nucleic acid residues, at least 90 nucleic acid residues, at least 100 nucleic acid residues, at least 125 nucleic acid residues, at least 150 nucleic acid residues, at least 175 nucleic acid residues, at least 200 nucleic acid residues, or at least 250 nucleic acid residues) of the nucleic acid sequence of said complete sequence.

The term ‘fragment of a polypeptide’ relates to peptides, oligopeptides, polypeptides, proteins, monomers, subunits and enzymes that comprise a stretch of contiguous amino acid residues, and exhibit substantially a similar, but not necessarily identical, functional or expression activity as the complete sequence. In a particular aspect, ‘fragment’ may refer to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of said complete sequence.

The term ‘polynucleotide’ means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more particularly 70 percent of its base pairs are in common, most particularly 90 per cent, and in a particular embodiment, 100 percent of its base pairs. The polynucleotides include polyribonucleic acids, polydeoxyribonucleic acids, and synthetic analogues thereof. It also includes nucleic acids with modified backbones such as peptide nucleic acid (PNA), polysiloxane, and 2′-O-(2-methoxy)ethylphosphorothioate. The polynucleotides are described by sequences that vary in length, that range from about 10 to about 5000 bases, particularly about 100 to about 4000 bases, more particularly about 250 to about 2500 bases. One polynucleotide embodiment comprises from about 10 to about 30 bases in length. A particular embodiment of polynucleotide is the polyribonucleotide of from about 17 to about 22 nucleotides, more commonly described as small interfering RNAs (siRNAs—both double stranded siRNA molecules and, self-complementary single-stranded siRNA molecules (shRNA)). Another particular embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection. Polynucleotides herein are selected to be ‘substantially’ complementary to different strands of a particular target DNA sequence. This means that the polynucleotides must be sufficiently complementary to hybridize with their respective strands. Therefore, the polynucleotide sequence need not reflect the exact sequence of the target sequence. For example, a non-complementary nucleotide fragment may be attached to the 5′ end of the polynucleotide, with the remainder of the polynucleotide sequence being complementary to the strand. Alternatively, non-complementary bases or longer sequences can be interspersed into the polynucleotide, provided that the polynucleotide sequence has sufficient complementarity with the sequence of the strand to hybridize therewith under stringent conditions or to form the template for the synthesis of an extension product.

The term “cancer” refers to a malignant or benign growth of cells in the blood, skin or in body organs, for example but without limitation, melanoma, lung, hematological malignancy, breast, prostate, lung, gastrointestinal, liver, neuroblastoma, glioblastoma, kidney, pancreas, stomach or bowel. A cancer tends to infiltrate into adjacent tissue and spread (metastasize) to distant organs, for example to bone, liver, lung or the brain. As used herein the term cancer includes both metastatic tumour cell types, such as but not limited to, melanoma, lymphoma, leukemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for prognostic or diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “binding”, “to bind”, “binds”, “bound” or any derivation thereof refers to any stable, rather than transient, chemical bond between two or more molecules, including, but not limited to, covalent bonding, ionic bonding, and hydrogen bonding. Thus, this term also encompasses hybridization between two nucleic acid molecules among other types of chemical bonding between two or more molecules.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

Mutations can be made in the sequences hereof such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following are examples of various groupings of amino acids:

Amino acids with nonpolar R groups: Alanine, Valine, Leucine, Isoleucine, Proline, Phenylalanine, Tryptophan, Methionine
Amino acids with uncharged polar R groups: Glycine, Serine, Threonine, Cysteine, Tyrosine, Asparagine, Glutamine
Amino acids with charged polar R groups (negatively charged at Ph 6.0): Aspartic acid Glutamic acid
Basic amino acids (positively charged at pH 6.0): Lysine, Arginine, Histidine (at pH 6.0)
Another grouping may be those amino acids with phenyl groups: Phenylalanine, Tryptophan, Tyrosine
Another grouping may be according to molecular weight (i.e., size of R groups):
Glycine (75), Alanine (89), Serine (105), Proline (115), Valine (117), Threonine (119), Cysteine (121), Leucine (131), Isoleucine (131), Asparagine (132), Aspartic acid (133),
Glutamine (146), Lysine (146), Glutamic acid (147), Methionine (149), Histidine (at pH 6.0) (155), Phenylalanine (165), Arginine (174), Tyrosine (181), Tryptophan (204)
Particularly preferred substitutions are: Lys for Arg and vice versa such that a positive charge may be maintained; Glu for Asp and vice versa such that a negative charge may be maintained;
Ser for Thr such that a free —OH can be maintained; and Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces—turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. Exemplary antibody molecules are intact immunoglobulin molecules, substantially intact immunoglobulin molecules and those portions of an immunoglobulin molecule that contains the paratope, including those portions known in the art as Fab, Fab′, F(ab)₂ and F(v), which portions are preferred for use in the therapeutic methods described herein. Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

An “antibody combining site” is that structural portion of an antibody molecule comprised of heavy and light chain variable and hypervariable regions that specifically binds antigen. The phrase “antibody molecule” in its various grammatical forms as used herein contemplates both an intact immunoglobulin molecule and an immunologically active portion of an immunoglobulin molecule. The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Therapeutically effective amount’ means that amount of a drug, compound, antimicrobial, antibody, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of infection of gram-positive bacteria, including having a bacteriocidal and/or bacteriostatic effect. The phrase “therapeutically effective amount” is used herein to mean an amount sufficient to prevent, and preferably reduce by at least about 30 percent, more preferably by at least 50 percent, most preferably by at least 90 percent, a clinically significant change in the growth or amount of infectious bacteria, or other feature of pathology such as for example, elevated fever or white cell count as may attend its presence and activity.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

“Attached” or “immobilized” as used herein to refer to a probe and a solid support may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

“Biological sample” as used herein may mean a sample of biological tissue or fluid that comprises nucleic acids. Such samples include fluid which is blood or serum, particularly serum. Such samples include, but are not limited to, tissue isolated from animals. Biological samples may also include sections of tissues such as biopsy and autopsy samples, frozen sections taken for histological purposes, blood, plasma, serum, sputum, stool, tears, mucus, urine, effusions, amniotic fluid, ascitic fluid, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

“Differential expression” may mean qualitative or quantitative differences in the temporal and/or cellular gene or RNA expression patterns within and among cells and tissue. Thus, a differentially expressed gene or RNA can qualitatively have its expression altered, including an activation or inactivation, in, e.g., normal versus disease tissue. Genes or RNA may be turned on or turned off in a particular state, relative to another state thus permitting comparison of two or more states. A qualitatively regulated gene or RNA will exhibit an expression pattern within a state or cell type that may be detectable by standard techniques. Some genes or RNAs will be expressed in one state or cell type, but not in both. Alternatively, the difference in expression may be quantitative, e.g., in that expression is modulated, up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. The term “expression profile” may also mean measuring the abundance of the nucleic acid sequences in the measured samples.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

As used herein, “pg” means picogram, “ng” means nanogram, “ug” or “μg” mean microgram, “mg” means milligram, “ul” or “μl” mean microliter, “ml” means milliliter, “l” means liter.

MicroRNAs (miRNAs) are ubiquitous regulators of biological processes involved in normal development, in differentiation and in diseases, including cancer. They act by regulating gene expression at the transcriptional and translational levels (Bartel, D. P. (2004) Cell 116:281-297). The regulation of gene expression by miRNAs is complex. Many mRNAs contain, within their 3′UTRs, binding sites for multiple miRNAs, and most miRNAs can potentially target a large number of genes (Bartel, D. P. (2004) Cell 116:281-297). It is clear that not every miRNA binding site predicted by sequence analysis contributes to a phenotype. Conversely, multiple miRNAs can affect a single cellular pathway (Mavrakis, K J et al (2008) Genes Dev 22(16):2178-2188; Li, Q-J et al (2007) Cell 29(1):147-161). miRNA expression analyses in various cancers have indicated that only a small number of miRNAs are highly expressed in cancer cells and that a pattern reminiscent of the tissue of origin is maintained (Lu, J et al (2005) Nature 435(7043):834-838; Landgraf, P et al (2007) Cell 129(7):1401-1414).

Early identification of recurrence risk and timely detection of relapse remains a clinical challenge in melanoma. The present invention and studies provided herein demonstrate the use of serum miRNAs in prognostic risk models to improve accuracy of early identification of primary melanoma patients at high risk for recurrence and as markers of melanoma recurrence.

In its primary aspect, the present invention concerns the identification of a miRNAs associated with cancer, particularly melanoma, and which have prognostic value or capability in predicting or indicating risk or likelihood or recurrence, and/or in predicting recurrence free survival (RFS) in melanoma and evaluation, assessment or monitoring thereof in the management and treatment of cancer. The present invention is thus based on the discovery that miRNA markers (including one or more of miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, including as set out below in Table 1, or otherwise as described and provided herein) disclosed herein may be utilized in the prognostic assessment of melanoma cancer. The present invention provides miRNA markers which are associated with melanoma cancer, particularly with recurrence, and uses of these miRNA markers in the evaluation, assessment and/or management or treatment of melanoma cancer. Exemplary miRNA markers are provided in TABLE 1 below:

TABLE 1
Serum Marker Homo sapiens (hsa) miRNAs

| | Exiqon | | |
|---|---|---|---|
| miRNA | Product ID | Seed Sequence | SEQ ID NO: |
| MARKERS | | | |
| miR-150/mir- | 204660 | UCUCCCAACCCUUGUACCAGUG | SEQ ID NO: 1 |
| 150-5p | | | |
| miR-15b/mir- | 204243 | UAGCAGCACAUCAUGGUUUACA | SEQ ID NO: 2 |
| 15b-5p | | | |
| miR-199a-5p | 204494 | CCCAGUGUUCAGACUACCUGUUC | SEQ ID NO: 3 |
| miR-33a/miR- | 204632 | GUGCAUUGUAGUUGCAUUGCA | SEQ ID NO: 4 |
| 33a-5p | | | |
| miR-423-5p | 204593 | UGAGGGGCAGAGAGCGAGACUUU | SEQ ID NO: 5 |
| miR-424/miR- | 204736 | CAGCAGCAAUUCAUGUUUUGAA | SEQ ID NO: 6 |
| 424-5p | | | |
| miR-let-7d/ | 204124 | AGAGGUAGUAGGUUGCAUAGUU | SEQ ID NO: 7 |
| miR-let-7d-5p | | | |
| miR-103/miR- | 204063 | AGCAGCAUUGUACAGGGCUAUGA | SEQ ID NO: 8 |
| 103a-3p | | | |
| miR-23b/miR- | 204790 | AUCACAUUGCCAGGGAUUACC | SEQ ID NO: 9 |
| 23b-3p | | | |
| miR-30d/miR- | 204757 | UGUAAACAUCCCCGACUGGAAG | SEQ ID NO: 10 |
| 30d-5p | | | |
| miR-425/miR- | 204337 | AAUGACACGAUCACUCCCGUUGA | SEQ ID NO: 11 |
| 425-5p | | | |
| miR-222 | 204551 | AGCUACAUCUGGCUACUGGGU | SEQ ID NO: 12 |
| miR-23a | 204772 | AUCACAUUGCCAGGGAUUUCC | SEQ ID NO: 13 |
| miR-26a | 204724 | UUCAAGUAAUCCAGGAUAGGCU | SEQ ID NO: 14 |
| miR-339-3p | 204160 | UGAGCGCCUCGACGACAGAGCCG | SEQ ID NO: 15 |
| miR-221/ | 204532 | AGCUACAUUGUCUGCUGGGUUUC | SEQ ID NO: 16 |
| miR221-3p | | | |
| NORMALIZERS | | | |
| miR-30c/miR- | 204783 | UGUAAACAUCCUACACUCUCAGC | SEQ ID NO: 17 |
| 30c-5p | | | |
| miR-181a/miR- | 204566 | AACAUUCAACGCUGUCGGUGAGU | SEQ ID NO: 18 |
| 181a-5p | | | |
| miR-142-3p | 204291 | UGUAGUGUUUCCUACUUUAUGGA | SEQ ID NO: 19 |
| miR-451/miR- | 204734 | AAACCGUUACCAUUACUGAGUU | SEQ ID NO: 20 |
| 451a | | | |
| miR-27b/miR- | 204782 | UUCACAGUGGCUAAGUUCUGC | SEQ ID NO: 21 |
| 27b-3p | | | |
| Quality Control | | | |
| miR-23a/miR- | 204772 | AUCACAUUGCCAGGGAUUUCC | SEQ ID NO: 22 |
| 23a-5p | | | |

The present invention relates to methods and compositions for the specific assessment and/or monitoring of expression and/or activity of one or more miRNAs particularly with reference to assessment of melanoma cancer, particularly associated with melanoma recurrence, including in combination, such miRNAs selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, including as set out in the Table above. It is herein demonstrated that assessment of microRNAs from Table 1 using reverse transcription methods to evaluate miRNA levels in a sample, particularly in serum, can be utilized alone or in combination with clinical covariates, such as tumor thickness, ulceration and/or anatomical site, to predict or indicate recurrence or determine likelihood or extent of recurrence free survival in a melanoma patient.

The prognostic possibilities that are raised by the recognition and understanding of the expression, level or activity of the miRNA markers(s) in serum in cancer patients, particularly melanoma patients, derive from the fact that the measurement or assessment of the miRNAs in serum appears to permit determination of or predict likelihood of recurrence in melanoma cancer. As suggested earlier and elaborated further on herein, the present invention contemplates determination and assessment of miRNA marker(s) in serum of a melanoma patient to provide prognostic information regarding the patient, including such as the patients likelihood of recurrence, or recurrence free survival and in monitoring for melanoma progression or relapse.

### Assays

The prognostic utility of the present invention extends to the use of the assessment of a combination of the miRNA markers(s) in cancer patients in assays to screen for or evaluate cancer, melanoma, recurrence, recurrence free survival in a mammal, particularly a human patient. The present invention thus includes an assay system which may be prepared in the form of a test kit for the analysis, including quantitative determination, of the extent of the presence of or the amount of a set of the miRNAs hereof. In an aspect thereof an assay is provided to screen to identify drugs or other agents that may mimic or block the expression or activity of the miRNA markers.

In addition, a method is provided for monitoring or evaluating the expression of two or more miRNAs associated with prognosis of recurrence of cancer in a patient with melanoma comprising the steps of:
(a) obtaining a sample of serum from a patient with melanoma;
(b) isolating RNA from the sample and amplifying RNA corresponding to at least three miRNA markers selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p; and
(c) detecting or measuring the expression or amount of said two or more miRNA markers in the sample,
   whereby the expression or amount of said miRNA markers is indicative of high or elevated risk of recurrence of cancer in the patient with melanoma.

In addition, a method is provided for monitoring or evaluating the expression of two or more miRNAs associated with prognosis of recurrence of cancer in a patient with melanoma comprising the steps of:
(d) obtaining a sample of serum from a patient with melanoma;
(e) isolating RNA from the sample and amplifying RNA corresponding to at least three miRNA markers selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p; and
(f) detecting or measuring the expression or amount of said two or more miRNA markers in the sample,
   whereby the expression or amount of said miRNA markers is indicative of recurrence of cancer in the patient with melanoma.

A method is provided for monitoring or evaluating the expression of miRNAs associated with prognosis of recurrence of cancer in a patient with melanoma comprising the steps of:
(g) obtaining a sample of serum from a patient with melanoma;
(h) isolating RNA from the sample and amplifying RNA corresponding to the miRNA markers miR-150, miR15b, miR-23b, and miR-425; and
(i) detecting or measuring the expression or amount of said miRNA markers in the sample,
   whereby the expression or amount of said miRNA markers is indicative of recurrence of cancer in the patient with melanoma. In a particular aspect, the expression or amount of said miRNA markers is compared to or normalized against the expression or amount of at least one normalization or control miRNA marker to determine if the marker miRNA expression or amount is indicative of recurrence of cancer in the patient with melanoma. In one such aspect the normalization or control miRNA marker is at least two selected from miR-142-3p, miR-451, miR-30c, miR-181a, miR-27b and miR-23a, particularly miR-30c and miR-181a.

In an aspect of the method, the expression or activity of said one or more miRNA is assessed by determining the amount of said miRNA, including by direct measurement or via specific reverse transcription of the miRNA, via quantitative PCR of the RNA, or using any other recognized or standard means to measure RNA amount or expression. Exemplary such methods are provided herein, including in the examples and otherwise are known to and/or within the capability of one skilled in the art. miRNA expression may be determined using, for example RT-PCR assays.

Cancer, including melanoma and probability or prediction of recurrence may be assessed or monitored by determining expression of two or more miRNAs, particularly selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p. In particular aspects recurrence is determined or prognosed by measuring and/or evaluating a collection or set of at least five miRNA markers in serum of a melanoma patient. A preferred set of serum miRNAs for analysis in a melanoma patient and with significance in methods, kits, assays to distinguish recurring from non-recurring melanoma patients or for prognosis of recurrence may be a set comprising miRNAs miR-150, miR-15b, miR-199-5p, miR-33a, miR-423-5p, miR-424, and miR-let-7d. A preferred set of serum miRNAs for analysis in a melanoma patient and with significance in methods, kits, assays to distinguish recurring from non-recurring melanoma patients or for prognosis of recurrence may be a set comprising miRNAs miR-150, miR-15b, miR-199-5p, miR-33a, and miR-424. A preferred set of serum miRNAs for analysis in a melanoma patient and with significance in methods, kits, assays to distinguish recurring from non-recurring melanoma patients or for prognosis of recurrence may be a set comprising miRNAs miR-103, miR-15b, miR-23b, miR-30d, miR-423-5p and miR-425. A preferred set of serum miRNAs for prognosis, recurrence or relapse analysis in a melanoma patient may be a set comprising miRNAs miR-103, miR-221, miR-222, and miR-423-5p. A preferred set of serum miRNAs for analysis in a melanoma patient and with significance in methods, kits, assays to distinguish recurring from non-recurring melanoma patients or for prognosis of recurrence may be a set comprising miRNAs miR-222, miR-23a, miR-26a, miR-339-3p and miR-423.5p. A particularly preferred set of serum miRNAs for analysis in a melanoma patient and with significance in methods, kits, assays to distinguish recurring from non-recurring melanoma patients or for prognosis of recurrence, including particularly for Stage I melanoma patients, may be a set comprising miRNAs miR-425/miR-425-5p, miR-150/miR-150-5p, miR-23b/miR-23b-3p and miR-15b/miR-15b-5p.

Serum miRNA sets may be provided for analysis in a melanoma patient, for example at Stage I, Stage II, or Stage III, with significance to prognose, predict or distinguish recurring from non-recurring melanoma. A preferred set of serum miRNAs for analysis in a melanoma patient with Stage III melanoma and with significance in methods, kits, assays to distinguish recurring from non-recurring Stage II melanoma patients or for prognosis of recurrence in a patient with diagnosed Stage II melanoma may be a set comprising miRNAs miR-30d, miR-199a-5p, miR-222, miR-423.5p and miR-424.

The invention thus includes a method for predicting recurrence of cancer, particularly melanoma, in a mammal comprising:

(a) obtaining a sample of blood or serum from said mammal;
(b) measuring the expression or amount of a set of miRNAs selected from:
(i) a set comprising miRNAs miR-150, miR-15b, miR-199-5p, miR-33a, miR-423-5p, miR-424, and miR-let-7d;
(ii) a set comprising miRNAs miR-103, miR-15b, miR-23b, miR-30d, miR-423-5p and miR-425;
(iii) a set comprising miRNAs miR-222, miR-23a, miR-26a, miR-339-3p and miR-423.5p.;
(iv) a set comprising miRNAs miR-30d, miR-199a-5p, miR-222, miR-423.5p and miR-424; and
(v) a set comprising miRNAs miR-425, miR-150, miR-15b and miR-23b;
   (c) comparing the expression or amount of said set of miRNAs to that in or from a reference sample or to the expression or amount of one or more normalization or control miRNAs;
   wherein the expression or amount of said set of miRNAs predicts or indicates the likelihood of recurrence of cancer in said mammal. In an aspect, the normalization or control miRNAs are selected from miR-142-3p, miR-451, miR-30c, miR-181a, miR-27b and miR-23a.

A method is contemplated and provided for evaluating melanoma cancer in a mammal comprising:

(a) obtaining a sample of blood or serum from said mammal;

(b) measuring the expression or amount of a set of miRNAs selected from:
(vi) a set comprising miRNAs miR-150, miR-15b, miR-199-5p, miR-33a, miR-423-5p, miR-424, and miR-let-7d;
(vii) a set comprising miRNAs miR-150, miR-15b, miR-199-5p, miR-33a, and miR-424;
(viii) a set comprising miRNAs miR-103, miR-15b, miR-23b, miR-30d, miR-423-5p and miR-425;
(ix) a set comprising miRNAs miR-222, miR-23a, miR-26a, miR-339-3p and miR-423.5p.;
(x) a set comprising miRNAs miR-30d, miR-199a-5p, miR-222, miR-423.5p and miR-424; and
(xi) a set comprising miRNAs miR-425, miR-150, miR-15b and miR-23b;
   (c) optionally comparing the expression or amount of said set of miRNAs to that in or from a reference sample or a reference or normal level or standard or to the expression or amount of one or more normalization or control miRNAs;
   wherein the expression or amount of said set of miRNAs is utilized to evaluate melanoma cancer and relative of risk of recurrence in said mammal.

In an aspect of the above methods, the expression or activity of two or more miRNAs selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p is measured and compared. In one aspect of the above methods, the expression or activity of three or more miRNAs is measured and compared. In one aspect of the above methods, the expression or activity of four or more miRNAs is measured and compared. In one aspect of the above methods, the expression or activity of five or more miRNAs is measured and compared. In one aspect of the above methods, the expression or activity of six or more miRNAs is measured and compared. In one aspect of the above methods, the expression or activity of seven or more miRNAs is measured and compared. In a further aspect, the cancer is selected from the group of skin cancer and melanoma.

The invention provides a method of monitoring melanoma progression and prognosticating an expected survival or recurrence in cancer, particularly melanoma, comprising the steps of: obtaining a biological sample, particularly serum or blood, from a subject in need of response or survival or recurrence prognostication; measuring an amount of at least one, at least two, at least three, at least four, at least five, at least six, at least seven, at least eight microRNA(s) in the biological sample, in particular selected from the microRNA(s) markers provided herein, in particular selected from Table 1, in particular selected from the group consisting of: miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, in particular one or more of the microRNA sets provided herein; wherein the amount of microRNA(s) found in the sample is prognostic for a low expected survival, is prognostic for recurrence of or in the subject; or is prognostic for an expected high survival, or is prognostic for a low probability of recurrence of or in the subject. In prognosis, the serum miRNA(s) amount may be optionally compared to an amount or amounts from standards or controls of appropriate nature, including extrapolated from normal or all cancer patients or historical amounts from mixed populations or samples. Exemplary micro RNA sets of use or relevant for the method(s) are provided herein and include a set of a set comprising miRNAs miR-150, miR-15b, miR-199-5p, miR-33a, miR-423-5p, miR-424, and miR-let-7d; a set comprising miRNAs miR-103, miR-15b, miR-23b, miR-30d, miR-423-5p and miR-425; a set comprising miRNAs miR-222, miR-23a, miR-26a, miR-339-3p and miR-423.5p.; and a set comprising miRNAs miR-30d, miR-199a-5p, miR-222, miR-423.5p and miR-424. In a particular and preferred aspect the method(s) relate to melanoma cancer.

Another aspect of the method of assessing cancer, prognosticating an expected response by a subject to a cancer treatment, or prognosticating risk or an expected survival or recurrence in cancer, particularly melanoma, of a subject comprises the following steps. A biological sample containing RNA from a subject in need of response or survival or recurrence prognostication is obtained. The biological sample is particularly and preferably serum. The biological sample is reacted with a reagent capable of binding to at least one, at least two, at least three, at least four, at least five, at least six, at least seven, at least eight microRNA(s) in the sample in particular selected from the microRNA(s) markers provided herein, in particular selected from Table 1, in particular selected from the group consisting of: miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, in particular one or more of the microRNA sets provided herein. The reaction forms a measurable microRNA(s). The amount of measurable microRNA(s) present in the sample or the expression profile of the measurable microRNA(s) present in the sample is then measured, and may optionally be compared to a standard or normalized amount of the microRNA(s) or the expression profile of the measurable microRNA(s) found in a normal cell or non-cancerous cell, or to an amount of the microRNA or the expression profile of the measurable microRNA(s) found in a control sample or mixed samples or normalized against all or any patients. A control sample may include one or more or many normal or diseased patient(s) or sample(s), including patient(s) with a different form of cancer or cancer or other disease history(ies). In an aspect, an altered level or expression of microRNA(s) in the sample relative to the standard(s) or control(s) indicates at least one of the following: the patient is at risk or not at risk for recurrence; the expected response by the subject to a cancer treatment is low or is high; recurrence in the patient is likely or unlikely; or the expected survival of the subject is low or high. In an aspect an altered level or expression of microRNA(s) in the sample relative to the standard(s) or control(s) indicates or prognoses that recurrence is likely in the patient or that the risk of recurrence is high or the recurrence free survival time in the patient is short or relatively short.

These microRNAs also relate to a composition to prognose, assess, or prognosticate a cancer. In one aspect, the composition comprises a compound capable of binding to at least a portion of a microRNA(s) selected from those provided herein, in particular at least one, at least two, at least three, at least four, at least five, at least six, at least seven, at least eight microRNA(s), in particular selected from the microRNA(s) markers provided herein, in particular selected from Table 1, in particular selected from the group consisting of: miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, in particular one or more of the microRNA sets provided herein. If the compound is bound to at least a portion of the microRNA(s), it forms or results in a measurable complex. A sample having an altered amount of the measurable complex is prognostic for a low expected response to the cancer treatment, is prognostic for a cancer recurrence, or is prognostic for a low expected survival, of the subject. Likewise, a sample having a normal amount of the measurable complex is prognostic for a high expected response to the cancer treatment, is prognostic for a low risk of or unlikely recurrence of cancer, or is prognostic for a high expected survival, by the subject.

Measuring the amount of microRNA(s) can be performed in any manner known by one skilled in the art of measuring the quantity of RNA within a sample. An example of a method for quantifying microRNA(s) is quantitative reverse transcriptase polymerase chain reaction. Another example of a method of quantifying microRNA(s) is as follows: hybridizing at least a portion of the microRNA(s) with a fluorescent nucleic acid, and reacting the hybridized microRN(s)A with a fluorescent reagent, wherein the hybridized microRNA(s) emits a fluorescent light. Another method of quantifying the amount of microRNA(s) in a sample is by hybridizing at least a portion of the microRNA(s) to a radio-labeled complementary nucleic acid. In instances when a nucleic acid capable of hybridizing to the microRNA(s) is used in the measuring step, the nucleic acid is at least 5 nucleotides, at least 10 nucleotides, at least 15 nucleotides, at least 20 nucleotides, at least 25 nucleotides, at least 30 nucleotides or at least 40 nucleotides; and may be no longer than 25 nucleotides, no longer than 35 nucleotides; no longer than 50 nucleotides; no longer than 75 nucleotides, no longer than 100 nucleotides or no longer than 125 nucleotides in length. The nucleic acid is any nucleic acid having at least 80% homology, 85% homology, 90% homology, 95% homology or 100% homology with any of the complementary sequences for the microRNAs selected from the group consisting of hsa-miR-15b, hsa-miR-181b, hsa-miR-191, hsa-miR-200c, hsa-let-7g, hsa-miR-425, hsa-miR-150 and hsa-miR23b.

The amount of microRNA(s) may be compared to either a standard amount of the microRNA(s) present in a normal cell or a non-cancerous cell, or to the amount of microRNA(s) in a control sample or the amount of microRNA(s) in a set, including a random set, of samples, whether cancer or non-cancer samples, and diseased or non-diseased. The comparison may be done by any method known to a skilled artisan. An example of comparing the amount of the microRNA(s) in a sample to a standard amount is comparing the ratio between 5S rRNA (or another standard or non-varying or ubiquitous RNA) and the microRNA(s) in a sample to a published or known ratio between 5S rRNA ( . . . ) and the microRNA(s) in a normal cell(s) or a non-cancerous cell(s) or random cell(s). An example of comparing the amount of microRNA(s) in a sample to a control is by comparing the ratios between 5S rRNA and the microRNA(s) found in the sample and in the control sample. In instances when the amount of microRNA(s) is compared to a control, the control sample(s) may be obtained from any suitable source, including known to have normal cells, cancer cells or non-cancerous cells. In an aspect, the control sample or normalizing sample(s) is tissue from the subject believed to contain only normal cells or non-cancerous cells or non-melanoma cells.

A biochip is also provided in accordance with the present invention. The biochip may comprise a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

In vivo animal models of cancer or animal xenograft studies may be utilized by the skilled artisan to further or additionally screen, assess, and/or verify the role of the miRNA(s) of the present invention and to assess, identify and characterize modulators of the miRNA(s), including antagomirs, of the present invention, including further assessing modulation of miRNA expression, modulation of miRNA target genes or proteins, and inhibiting cancer progression, growth, recurrence, metastasis, resistance and/or infiltration. Suitable animal models include, but are not limited to models of various cancers and hyperproliferative conditions. Any suitable cancer model may be utilized.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The miRNA Target(s) or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

In a further embodiment of this invention, commercial test kits suitable for use by a medical specialist may be prepared to determine the presence or absence of predetermined miRNA activity or capability in suspected target or cancer cells. In accordance with the testing techniques discussed above, one class of such kits will contain at least the labeled miRNA or its binding partner, for instance a tumor suppressor gene, and directions, of course, depending upon the method selected, e.g., “competitive,” and the like. The kits may also contain peripheral reagents such as buffers, stabilizers, etc.

### Oligonucleotides

The present invention provides an antagomir, oligonucleotide, nucleic acid which is substantially complementary to one or more miRNA marker(s), particularly miRNA selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p, wherein said oligonucleotide alters the expression or activity of one or more miRNA marker(s). Exemplary antagomirs may be determined and assessed by one of skill in the art based on the sequences of the miR(s) seed sequence as set out in TABLE 1 and are complementary thereto.

Exemplary antagomirs or oligonucleotides may be prepared synthetically or may be available commercially, such as as miRZIPs™ from System Biosciences (Mountain View, Calif.; systembio.com). Stoffel and colleagues first described silencing of miRNAs in vivo with “antagomirs” in 2005 (Krutzfeldt J et al (2005) Nature 438(7068):685-689). Intravenous administration of antagomirs against specific miRNAs resulted in marked reduction of corresponding miRNA levels in specific organs. Chemically modified single-stranded RNA analogues complementary to a specific miRNA are effective as antagomirs. Oligonucleotides have been described linked to cholesterol molecule to enhance uptake and improve target degradation and with phosphorothioate modifications (Krutzfeld J et al (2007) Nucl Adids Res 35(9):2885-2892). Pharmaceutical compositions of shortened and modified oligonucleotide antagomirs have been described including in US2010/0286234 and WO 2010/144485 A1. Scherr et al have described lentivirus-mediated antagomir expression systems for specific inhibition of miRNA function (Scherr M et al (2007) Nucl Acids Res 35(22):e149 (doi:10.1093/nar/qkm971). Lentivirus antagomirs are available commercially, including as miRZIPs™ from System Biosciences, as well as microRNA mimics meridian from Dharmacon/ThermoScientific. The knowledge of miRNA sequences (including publicly available in databases such as mirBase) and commercial and public availability of antagomirs, lentivirus constructs, and synthetic oligonucleotides makes antagomirs/oligonucleotides as exemplary miRNA Target(s) inhibitors available for testing, assessment and evaluation. Thus, one of skill in the art can readily design, make or acquire suitable antagomirs or oligonucleotides for use and application in accordance with the present invention.

The invention includes an oligonucleotide, probe, antisense oligonucleotide or an antagomir comprising a sequence substantially complementary to at least one of the miRNA sequences provided herein, set out in Table 1, or one or more selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p. The antagonist or antagomir may be substantially complementary to the miRNA target sequences set out in Table 1. In particular, antagomirs, antagonists or oligonucleotides of the invention include oligonucleotides comprising a sequence substantially complementary to nucleotides selected from the group of SEQ IDs 1-15 as set out in Table 1, or a subset of nucleotides thereof sufficient to monitor or alter the expression or activity of one or more of said miRNA sequences SEQ ID NOs 1-15. The invention includes antisense oligonucleotides or antagomirs comprising one or more sequence complementary to one or more sequences out in Table 1, provided herein, or complementary to sequence(s) set out in SEQ ID NOS: 1-15

The one or more nucleic acid or oligonucleotide of the invention may comprise at least one modified nucleotide. In a particular aspect, the nucleic acids, probes, oligonucleotides, antagomirs of the present invention or of use in the present invention may be modified, either by manipulation of the chemical backbone of the nucleic acids or by covalent or non-covalent attachment of other moieties. In each or any case, such manipulation or attachment may serve to modify the stability, cellular, tissue or organ uptake, or otherwise enhance efficacy of the nucleic acids and oligonucleotides. In further aspects of the invention, the nucleic acids, probes, oligonucleotides or antagomirs may be covalently linked to other molecules, including but not limited to polypeptides, carbohydrates, lipid or lipid-like moieties, ligands, chemical agents or compounds, which may serve to enhance the uptake, stability or to target the oligonucleotides. The nucleic acids, probes, oligonucleotides or antagomirs of the present invention may be combined with oligonucleotides directed to or specific for other targets or markers, by mixture or by non-covalent or covalent attachment.

The skilled artisan can readily utilize any of several strategies to facilitate and simplify the selection process for nucleic acids, probes and oligonucleotides effective in determining expression or activity of one or more miRNA marker(s), including as selected from miR-150, miR15b, miR-199a-5p, miR-33a, miR-423-5p, miR-424, miR-let-7d, miR-103, miR-23b, miR-30d, miR-425, miR-222, miR-23a, miR-26a, and miR-339-3p. Predictions of the binding energy or calculation of thermodynamic indices between an oligonucleotide and a complementary sequence in an mRNA molecule may be utilized (Chiang et al. (1991) J. Biol. Chem. 266:18162-18171; Stull et al. (1992) Nucl Acids Res. 20:3501-3508). Oligonucleotides may be selected on the basis of secondary structure (Wickstrom et al (1991) in Prospects for Antisense Nucleic Acid Therapy of Cancer and AIDS, Wickstrom, ed., Wiley-Liss, Inc., New York, pp. 7-24; Lima et al. (1992) Biochem. 31:12055-12061). Schmidt and Thompson (U.S. Pat. No. 6,416,951) describe a method for identifying a functional antisense agent comprising hybridizing an RNA with an oligonucleotide and measuring in real time the kinetics of hybridization by hybridizing in the presence of an intercalation dye or incorporating a label and measuring the spectroscopic properties of the dye or the label's signal in the presence of unlabelled oligonucleotide. In addition, any of a variety of computer programs may be utilized which predict suitable probe, oligonucleotide or antagomir sequences utilizing various criteria recognized by the skilled artisan, including for example the absence of self-complementarity, the absence of hairpin loops, the absence of stable homodimer and duplex formation (stability being assessed by predicted energy in kcal/mol). Examples of such computer programs are readily available and known to the skilled artisan and include the OLIGO 4 or OLIGO 6 program (Molecular Biology Insights, Inc., Cascade, Colo.) and the Oligo Tech program (Oligo Therapeutics Inc., Wilsonville, Oreg.).

“Substantially complementary” is used to indicate a sufficient degree of complementarity such that stable and specific binding occurs between the DNA or RNA target and the oligonucleotide or nucleic acid. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility or expression, and there is a sufficient degree of complementarity to avoid non-specific binding of the oligonucleotide to non-target sequences under physiological conditions in the case of in vivo assays or therapeutic treatment or, in the case of in vitro assays, under conditions in which the assays are conducted.

In the context of this invention, the term “oligonucleotide” refers to an oligomer or polymer of nucleotide or nucleoside monomers consisting of naturally occurring bases, sugars and intersugar (backbone) linkages. Oligonucleotide includes oligomers comprising non-naturally occurring monomers, or portions thereof, which function similarly and such modified or substituted oligonucleotides may be preferred over native forms because of, for example, enhanced cellular uptake and increased stability against nucleases. The oligonucleotides of the present invention may contain two or more chemically distinct regions, each made up of at least one nucleotide, for instance, at least one region of modified nucleotides that confers one or more beneficial properties (for example, increased nuclease resistance, increased uptake into cells, increased binding affinity for the RNA target) and a region that is a substrate for enzymes capable of cleaving RNA:DNA or RNA:RNA hybrids (for example, RNase H—a cellular endonuclease which cleaves the RNA strand of an RNA:DNA duplex).

Oligonucleotides or antagomirs may also include, additionally or alternatively base modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-me pyrimidines, particularly 5-methylcytosine (5-me-C) (Sanghvi, Y. S., in Crooke, S. T. and Lebleu, B., eds., Antisense Research and Applications, CRC Press, Boca Raton, 1993, pp. 276-278), 5-hydroxymethylcytosine (HMC), glycosyl HMC and gentobiosyl HMC, as well as synthetic nucleobases, including but not limited to, 2-aminoadenine, 2-thiouracil, 2-thiothymine, 5-bromouracil, 5-hydroxymethyluracil, 8-azaguanine, 7-deazaguanine (Kornberg, A., DNA Replication, W.H. Freeman & Co., San Francisco, 1980, pp 75-77; Gebeyehu, G., et al., 1987, Nucl. Acids Res. 15:4513). A “universal” base known in the art, e.g., inosine, may be included.

### Compositions

The present invention further contemplates compositions, including prognostically and therapeutically relevant compositions, useful in practicing the methods of the invention. A subject therapeutic composition includes, in admixture, a pharmaceutically acceptable excipient (carrier) and one or more of an antagomir, oligonucleotide, miRNA marker antagonist, as described herein as an active ingredient. In a preferred embodiment, the composition comprises an agent capable of determining or monitoring miRNA amounts or expression (for instance by binding to the miRNA(s)) or of modulating the specific binding of the miRNAs of the present invention to their target(s) within a target cell, particularly a cancer cell or pre-cancerous cell.

The preparation of prognostic or therapeutic compositions which contain nucleic acids, antagomirs, oligonucleotides or miRNA antagonists as active ingredients is well understood in the art. Such therapeutic compositions may be prepared as injectables, either as liquid solutions or suspensions, however, solid forms suitable for solution in, or suspension in, liquid prior to injection can also be prepared. The preparation can also be emulsified. The active therapeutic ingredient is often mixed with excipients, additives, labels which are prognostically, diagnostically and/or pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An antagomir(s), oligonucleotide(s) or miRNA antagonist(s) can be formulated into the therapeutic composition as neutralized pharmaceutically acceptable salt forms. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

As is well known in the art, DNA sequences may be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the nucleic acid sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the nucleic acid sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular nucleic acid sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Synthetic DNA/RNA sequences allow convenient construction of genes which will express miRNA analogs or “muteins”. Alternatively, DNA encoding muteins can be made by site-directed mutagenesis of native RNA, genes or cDNAs, and muteins can be made directly using conventional polypeptide synthesis. A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, 1988.). Because they are sequence-specific, only mRNAs with particular sequences are inactivated. Investigators have identified two types of ribozymes, Tetrahymena-type and “hammerhead”-type. (Hasselhoff and Gerlach, 1988) Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Serum MicroRNAs as Prognostic Biomarkers for Recurrence in Melanoma

Early identification of recurrence risk and timely detection of relapse remains a clinical challenge in melanoma. These studies demonstrate support for the use of serum miRNAs in prognostic risk models to improve accuracy of early identification of primary melanoma patients at high risk for recurrence and as markers of melanoma recurrence.

Due to the heterogeneity of melanoma outcomes unaccounted for by the current staging system, early and accurate identification of patients at higher risk of recurrence remains a challenge. We investigated the prognostic potential of serum microRNAs (miRNAs) in predicting recurrence of primary melanoma patients at the time of diagnosis. Using a qPCR panel containing 355 miRNAs, we screened the serum of melanoma patients drawn at the time of diagnosis to identify miRNAs with predictive potential. Using multivariate modeling, we identified miRNA signatures that separated patients into high and low recurrence risk groups independent of stage. These results were validated in an independent cohort. Further refinement of these miRNA based prognostic models can result in the development of clinical assay that can be used to better inform clinical decision making by improving patient stratification.

Purpose:

Identification of primary melanoma patients at high risk for recurrence is critical for informed management decisions. We hypothesized that serum miRNAs could provide prognostic information at the time of diagnosis not accounted for by the current staging system and could be useful in detecting recurrence in resected melanoma patients.

Experimental Design:

We screened 355 miRNAs in sera from 80 melanoma patients at the time of primary diagnosis (discovery cohort) using a unique quantitative reverse transcription-PCR (qRT-PCR) panel. Cox proportional hazard models and Kaplan-Meier recurrence-free survival (RFS) curves were used to identify a miRNA signature with prognostic potential adjusting for stage. Stage-weighted multivariable logistic regression models were fitted with adjustment for tumor thickness. miRNA signatures were selected based on optimal classification or predictive potential (area under the ROC curves, AUCs), and then assessed in an independent cohort of 50 primary melanoma patients (validation cohort). Selected miRNAs were also measured longitudinally in a subset of patients pre- and post-operatively and pre- and post-recurrence.

Results:

Multiple logistic regression models using selected miRNAs were able to separate the discovery cohort into a high recurrence risk group with significantly shorter recurrence free survival (RFS) compared to a low risk group (p<0.005). The ability to distinguish high versus low recurrence risk was also observed in the validation cohort (p<0.005). Using the discovery cohort, subset analysis of Stage II patients showed that a model incorporating 5 miRNAs yielded an AUC of 80% compared to 55% using thickness alone. The same model yielded an AUC=96% (95% CI, 89-100%) when assessed in the validation cohort, and was able to separate Stage II patients into a shorter RFS group versus a longer RFS group (p<0.01). We identified two signatures of 5 miRNAs that successfully classified recurred from non-recurred patients at the time of diagnosis in cohort 1 (AUC=0.86 and 0.89, respectively). The miRNA-based risk models successfully classified primary melanoma patients into high and low recurrence risk groups with significant separation of recurrence free survival. The classification performance was maintained when models were evaluated in cohort 2 (AUCs>0.96). Longitudinal expression of 4 miRNAs was dynamic, suggesting miRNAs can be associated with tumor burden.

Conclusion:

Our data demonstrate that serum miRNAs can be incorporated into prognostic models and have potential clinical utility as signatures in prognostic risk models to improve accuracy in identifying primary melanoma patients with high recurrence risk at the time of diagnosis and in monitoring tumor burden over time.

Melanoma remains a highly morbid disease in the United States and its incidence has continued to rise sharply over the past few decades (1). In addition, the toll of melanoma in terms of “life-years lost” is the highest of all solid tumors in the United States (2). Recurrence risk varies by stage, with estimated recurrences rates of up to 30% for localized melanoma and 60% of regional nodal disease (3). In the absence of curative therapy for patients with metastatic melanoma, the identification of patients at high risk for recurrence at the time of primary diagnosis is critical for informed management decisions.

The current standards of care for determining prognosis of melanoma patients with localized disease and guiding post-operative follow up has limitations. Using prognostic factors, such as Breslow depth, primary ulceration, mitotic rate and lymph node involvement, the current AJCC staging system is able provide first line stratification for melanoma specific survival (4). However, the current staging system only partly explains the variability in the prognosis of melanoma and there remains unexplained heterogeneity within each stage. Additionally, despite the benefits of early detection of loco-regional and distant metastasis amenable to curative resection (3,5), there is no consensus on selection and timing of imaging studies and laboratory tests for use in follow-up and existing guidelines are not consistently applied (6). This is in part due to the limited sensitivity and specificity of available imaging modalities and blood tests, coupled with considerable economic cost (7).

Due to the routine collection and facility of obtaining blood samples at multiple time points, blood-based biomarkers are a logical and cost effective source in the search for non-invasive biomarkers. Although assessment of circulating markers for prognosis and surveillance has been part of the standard of care in breast and colon cancer management for several years (8), no such markers exist for melanoma. While many molecules have been studied as biomarkers for use in melanoma (9-11), none have been developed into a clinically relevant assay. Currently, serum lactate dehydrogenase is the only blood-based marker routinely used in melanoma, but has only shown prognostic significance in advanced disease (4, 10, 11) and is rarely the sole indicator of recurrence (12). There remains a need to develop methods to accurately determine recurrence risk of primary melanoma patients and improve follow-up for early detection of relapse.

MicroRNAs (miRNAs) are small, non-coding RNAs that negatively regulate gene expression at the post transcriptional level (13). Given their implication in tumorigenesis, tissue-specific dysregulation in cancer (14), and present in human serum in an extremely stable form resistant to RNase digestion, harsh conditions, extended storage, and multiple freeze-thaw cycles (15), miRNAs have emerged as a novel source of blood-based reporters of cancer progression. Studies assessing serum or plasma miRNAs as biomarkers in a variety of cancers have largely focused on distinguishing cancer patients from control subjects (16,17). While there is a growing interest in evaluating the prognostic potential of circulating miRNAs (18-21), to the best of our knowledge, no study has specifically examined the association between serum-based miRNAs and recurrence risk in primary melanoma patients. We examine the prognostic utility of combining sera miRNAs and clinical characteristics, at the time of primary diagnosis, in identifying melanoma patients at high risk of disease recurrence. In this study, we demonstrate the prognostic value of incorporating a set of serum miRNAs into a model with clinicopathological covariates in predicting recurrence of primary melanoma patients.

Methods

Study Population

We studied primary cutaneous melanoma patients (140 patients) with AJCC stage I-III disease prospectively enrolled in a melanoma database at New York University (NYU) Langone Medical Center. Serum was collected from patients at the time of diagnosis, prior to treatment. A discovery cohort of 80 patients (cohort 1) and an independent cohort 50 patients (cohort 2) were used to construct prognostic models. Sera collected at the time of primary diagnosis and recurrence were available for a subset of patients (n=17). All patients had histologically confirmed melanoma (22). Relevant demographic and clinicopathological data were obtained from a prospectively maintained NYU Interdisciplinary Melanoma Cooperative Group (IMCG) database (22) and are listed in TABLE 2. Median time of follow-up for survivors was 42.5 months (range, 17-237 months). Both loco-regional (stage III) and distant metastases (stage IV) were defined as recurrences. Recurrence status by stage is shown in TABLE 3. Additionally, an independent set of 10 melanoma patients had sera collected before and 7-14 days after surgical resection of tumors. Patients with melanoma-in-situ were excluded from study. The study was approved by the NYU Institutional Review Board and informed consent was obtained from all patients.

TABLE 2
Baseline characteristics of melanoma patients

| | Melanoma patients |
|---|---|
| | (n = 140) |
| | N (%) |
| Age at diagnosis, years | |
| Median | 59 |
| Gender | |
| Male | 82 (59) |
| Female | 58 (41) |
| Thickness, mm | |
| median (range) | 2 (0.27-28) |
| Ulceration | |
| Present | 49 (35) |
| Absent | 90 (64) |
| Mitosis | |
| Absent | 27 (19) |
| Present | 96 (69) |
| Unclassified | 17 (12) |
| Histological type | |
| Superficial Spreading | 54 (39) |
| Nodular | 44 (31) |
| Other^{a} | 25 (18) |
| Unclassified | 17 (12) |

NOTE.
Percentages may not sum to 100% as a result of rounding
^{a}Other includes acral lentiginous melanoma, desmoplastic melanoma and lentigo maligna

TABLE 3
Recurrence Status of Melanoma Patients Stratified by Stage

| | Cohort 1 | | Cohort 2 | |
|---|---|---|---|---|
| | Non-recurrence | Recurrence | Non-recurrence | Recurrence |
| Stage I | 34 | 5 | 10 | 0 |
| Stage II | 13 | 7 | 16 | 12 |
| Stage III^{a} | 8 | 13 | 4 | 8 |
| Totals | 55 | 25 | 30 | 20 |

^{a}Includes 1 patient diagnosed with stage III melanoma, but did not have blood drawn until time of recurrence with stage IV disease

Serum Preparation and miRNA Extraction

All serum samples were collected in 10-mL BD serum tubes, stored immediately at 4° C., and then centrifuged at 10° C. for 10 minutes at 1,500×g. The supernatant serum was then aliqouted into 1.5 mL cryovials and stored at −80° C. until further use. Total RNA was extracted from serum using the Qiagen miRNeasy® Mini Kit using manufacturer's instructions with minor modifications. Notably, serum was thawed on ice and centrifuged at 3000×g for 5 min in a 4° C. microcentrifuge. Aliquots of 200 μL of serum per sample were transferred to a new microcentrifuge tube and 750 μl of a Qiazol mixture containing 1.25 μg/mL of MS2 bacteriophage RNA was added to the serum Total RNA was eluted by adding 50 μL of RNase-free water to the membrane of the Qiagen RNeasy® mini spin column and incubating for 1 min before centrifugation at 15,000×g for 1 min at room temperature. The RNA was stored in a −80° C. freezer.

miRNA Real-Time qPCR Discovery

Reverse transcription (RT) was performed using 8 μl RNA from the total eluate of 50 μl in 40 μl reactions using the miRCURY LNA™ Universal RT microRNA PCR, and Polyadenylation and cDNA synthesis kit (Exiqon, Denmark). cDNA was diluted 50× and assayed in 10 μl PCR reactions according to the protocol for miRCURY LNA™ Universal RT microRNA PCR; each microRNA was assayed by qPCR on a discovery panel containing 355 miRNA assays and positive and negative controls. Twenty-two of the miRNA assays are present in two versions with difference in specificity within miRNA family and sensitivity. Negative controls excluding template from the reverse transcription reaction were performed and profiled like the samples. The amplification was performed in a LightCycler® 480 Real-Time PCR System (Roche) in 384 well plates. The amplification curves were analyzed using the Roche LC software, both for determination of Ct (by the 2nd derivative method) and for melting curve analysis. Only assays detected with 5 Ct's less than the negative control and with Ct<40 were included in the data analysis. Expression data that did not pass the quality control (QC) criteria were not included in further analysis.

The qPCR platform utilized (Exiqon) has been demonstrated to be the most sensitive and specific qPCR platform available when analyzing ultra low miRNA levels (200 copies or less) as is found in serum and other biofluid samples (23). The miRCURY platform has a very high degree of linearity (r²≧0.9) across four log scales of miRNA copies for all assays. True sensitivity of the platform is maximized because of two features. First, cDNA synthesis is carried out using a universal approach that allows all miRNAs in a sample to be synthesized equally. Second, LNAs are incorporated into the PCR primers making the PCR assay performance independent of GC content, which can otherwise affect the miRNAs ability to be efficiently amplified and accurately quantified. For this study, a unique Pick & Mix serum discovery panel consisting of 355 miRNAs was developed based on results of a genome-wide qPCR analysis of more than 2,000 healthy individuals and patients with numerous cancers including colon, breast, lung, melanoma, pancreatic, head and neck as well as different inflammatory diseases, hypertension and diabetes.

miRNA Real-Time qPCR Validation

miRNAs identified as predictors of recurrence in multivariable models with adjustment for tumor stage and thickness were selected for validation using individual qRT-PCR assays. Following the same protocol described above, two replicate RT's were set up for each sample, this time using 2 μl RNA in 10 μl reactions. Each miRNA was assayed by qPCR once for each RT. Negative controls excluding template from the reverse transcription reaction were performed and profiled like the samples. Amplification and analysis were performed as described above.

Of the 355 miRNA assays, 170miRNAs were included in analysis as they had Ct<40 and were detected with 5Cts lass than the no RT template included negative control in greater than two-thirds of patients. Expression data for 2 patients did not pass the quality control (QC) criteria and were not included in further analysis. Additionally, 1 patient had missing expression data for miR-199a-5p and was not included in analyses of models containing this miRNA.

A panel of 11 miRNAs (mir-15b, -23b, -30d, -33a, -103, -150, 199a-5p, -423-5p, -424, -425 and let-7d) identified as potential predictors of recurrence in multivariate models were selected for evaluation in the validation cohort using individual qRT-PCR assays.

Longitudinal Evaluation of miRNA Expression

For evaluation of pre/post-operative and pre/post recurrence samples, we selected 7 miRNAs (miR-103, -182, -191, -221, -222, -423-5p, and -425) identified by our analyses and in tissue-based miRNA studies as associated with progression (23, 32).

In exploring the potential of serum miRNAs as markers of melanoma recurrence, we elected to use a targeted approach to select miRNAs for study in pre-/post-operative and pre-/post-recurrence samples. We included several of the miRNAs that we identified as having prognostic potential in our discovery phase and were further evaluated in the validation cohort (i.e. miR-103, -191, -423-5p, -425). We did not limit ourselves to using only those miRNAs included in the recurrence risk signature as markers of prognosis may not translate into markers of disease detection. Instead, we focused on miRNAs from our prioritized panel that had previously supported roles in cancer progression and/or diagnostic utility. For example, high levels of miR-103 are associated with metastasis and poor outcome in breast cancer patients and functionally, mir-103 confers migratory capacities in vitro [24]. Elevated expression of miR-191 promotes epithelial-to-mesenchymal transition in hepatocellular carcinoma [25]. miR-423-5p was identified as part of a 5-miRNA signature for gastric cancer diagnosis [26]. We also chose to include one miRNA, miR-425, whose functional relevance in cancer was previously unexplored to avoid eliminating potentially useful markers. Additionally, we selected miRNAs that have well supported roles in melanoma progression (i.e. miR-182, -221, -222) as demonstrated by tissue-based studies [27, 28].

Total RNA was extracted using the miRVana Paris isolation system (Ambion, USA) following the manufacturer's protocol with the addition of an acid/phenol/chloroform extraction in a final elute of 100 μl . RT was performed using 2.5 μl miRNA in a final volume of 10 μl following manufacturer's instructions using TaqMan MicroRNA Reverse Transcription kit (Applied Biosystems, USA). qRT-PCR was performed in triplicate on a MyIQ Single Color Real Time PCR detection system (Bio-Rad, USA), using 1.341 cDNA, 1 μl miRNA-specific TaqMan® primer and 1× Hotmaster Master mix containing Taq DNA polymerase (5 Prime, USA) and dNTP Mix (Promega, USA) in a final volume of 20 μl per reaction. The amplification protocol was: 95° C. for 17 min, 40 cycles at 95° C. for 15 sec followed by 60° C. for 1 min.

Statistical Methods

Using the discovery cohort (n=80), miRNAs were first ranked by univariate association of expression level for each miRNA with recurrence-free survival (RFS) via Cox proportional hazards regression analysis with adjustment for tumor stage. Top-ranking miRNAs were used as candidates to be included in the multivariate Cox proportional hazards model. The 5 miRNA-signature was selected by minimizing Akaike's information criterion (AIC) of the multivariate Cox PH model through stepwise selection[29,30]. The linear combination of model predictors weighted by regression coefficients was defined as the risk score. A cutoff of the risk score was chosen to separate patients into high and low recurrence risk groups [31]. Kaplan-Meier survival curves for the resulting groups were plotted, and log-rank test was used to compare the two curves. To test the classifier, regression coefficients of the Cox model were applied to the validation cohort (n=50) to obtain a risk score, and the same cutoff was used to predict RFS in the validation cohort.

The identified miRNA signature set was also evaluated for its utility in predicting 3-year RFS by logistic regression model using recurred patients (n=25) and non-recurred patients (n=44) with ≧3 years follow-up from the discovery cohort as cases and controls, respectively [32]. An optimal risk score cutoff using the Youden Index of the Receiver Operating Characteristic (ROC) curve was chosen to classify patients into high and low risk groups. Kaplan-Meier survival curves and log-rank tests were used to compare the RFS distributions of the two groups. The logistic model was used to predict recurrence risk in the validation cohort of 20 recurred and 16 non-recurred patients with ≧3 years follow-up. The same risk score cutoff was used to classify patients.

As a subset analysis to demonstrate the utility of serum miRNAs beyond tumor stage, we built a logistic model using the panel of 11 prioritized miRNAs in all stage II patients and examined its ability to distinguish patients with and without recurrence by cross validation, and examined the RFS difference using Kaplan-Meier curves between the predicted high vs. low risk groups. Longitudinal changes in miRNA expression were assessed using two-sided Student's t tests (p<0.05). All statistical analyses were performed in R 2.12.0.

The reported results are based on Ct values that passed QC but without further normalization. To assess the method for analysis, various ways of normalizing miRNA expression were explored (i.e. median normalization: shift Ct values on each panel by additive constants such that the medians of miRNA expression are the same across panels). The resulting candidate set of miRNAs identified were similar to those obtained when data were not normalized; moreover, selected classifiers worked well whether Ct values were normalized or not, indicating the robustness of the developed models.

Results

miRNA Profiling in Serum Identifies miRNAs with Prognostic Potential

Serum samples from 80 patients with primary melanoma were initially tested as discovery cohort or cohort 1. Median time of follow-up was ≧3 years (38 months) for survivors. In the validation cohort (cohort 2) of 50 patients, 5 samples were excluded (2 due to poor specimen quality, 2 with unknown tumor thickness, and 1 with no expression of one of the candidate miRNAs). Baseline characteristics of both cohorts are illustrated in TABLE 1.

For the development of logistic risk models, non-recurrence was defined as no evidence of disease with at least three years of follow-up. First, using data from the discovery cohort of 80 patients, stage-weighted discriminative models were explored using clinical covariates alone as predictors of recurrence. Thickness, ulceration, and anatomic site were examined. Tumor thickness had the smallest p-value compared to other clinical covariates, and was therefore included in subsequent risk models. Next, we identified candidate miRNAs from the discovery cohort data using three small logistic regression models.

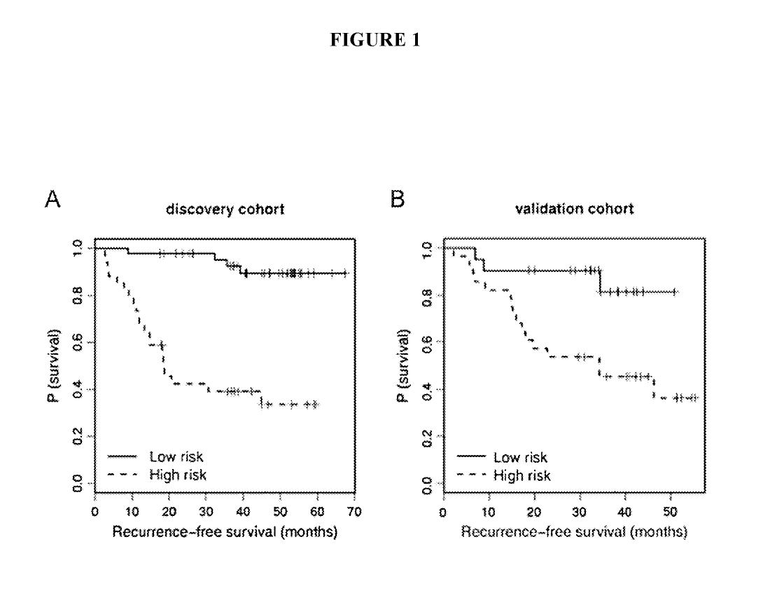

The first model built from the discovery cohort used a set of 7 miRNAs (miR-150, -15b, -199a-5p, -33a, -423-5p, -424, -let-7d) and, when adjusting for thickness, achieved an AUC=85% under the ROC curve when used to classify recurred from non-recurred patients. The Youden index of the ROC was used as a cut-off value to classify the discovery cohort into high and low recurrence risk groups. The Kaplan-Meier survival curves (FIG. 1A) for the recurrence-free-survival (RFS) indicated significant differences in distribution between the high and low risk groups (log-rank test, p<0.001). To assess the utility of these 7 miRNAs, they were fitted in the validation cohort with adjustment for thickness and yielded a statistically significant stage-weighted logistic model (p=0.004). The resulting ROC had an AUC=94%, and when using a cut-off value (sensitivity=100%, specificity=74%) to classify the validation cohort into high and low risk groups, the two groups have significantly different RFS (log-rank test, p=0.0005, FIG. 1B).

A first model subset of 5 miRNAs (miR-150, -15b, -199a-5p, -33a, -424) (denoted Subset Model 1) also showed promise as recurrence signature. This model was significant (p=0.0001, TABLE 4) and showed clear classification performance (AUC=0.86) when used to classify recurred from non-recurred patients. When this subset model was used to separate all of cohort 1 into high and low recurrence risk groups using an optimal cut-point (sensitivity=76%, specificity=81%), recurrence free survival (RFS) of the two groups had clear separation (data not shown).

We screened serum samples of the discovery cohort using a qPCR platform of 355 miRNAs, of which data for 170 miRNAs were available for at least 67% samples. A multivariate Cox PH model for RFS was identified, which contains 5 miRNAs (miR-150, -15b, -199a-5p, -33a, -424) with adjustment for stage (TABLE 4). The linear combination of model predictors weighted by regression coefficients was defined as the risk score. Motivated by Satzger, et al. [31], a cutoff was chosen to separate patients into high and low recurrence risk groups aimed at maximizing the log-rank statistic (sensitivity=0.84, specificity=0.76; high risk, n=34, low risk, n=46). Kaplan-Meier analysis revealed that the two groups have a significant separation in RFS (p=0.0036, FIG. 1A). The model was applied to predict recurrence risk in the validation cohort, and the same cutoff was used to separate patients into high and low recurrence risk groups (sensitivity=0.84, specificity=0.60; high risk n=28, low risk n=21). Kaplan-Meier analysis indicated that the resulting RFS distributions were again significantly different (p=0.009, FIG. 1B).

TABLE 4
Covariates Included in Multivariate
Cox Proportional Hazards Models

| Covariate | p-value | HR (95% CI) |
|---|---|---|
| Stage II | 0.0108 | 4.862 (1.442-16.397) |
| Stage III | 4.1e−05 | 9.366 (3.125-27.287) |
| miR-150 | 0.1469 | 1.297 (0.913-1.843) |
| miR-15b | 0.0159 | 0.437 (0.223-0.856) |
| miR-199a-5p | 0.1383 | 1.375 (0.903-2.094) |
| miR-3a | 0.1099 | 0.720 (0.481-1.077) |
| miR-424 | 0.0094 | 1.821 (1.158-2.862) |

Abbreviation: HR: Hazard ratio; CI: confidence interval

Logistic Regression Analysis Confirms Prognostic Potential of miRNA-Based Risk Model for 3-Year RFS

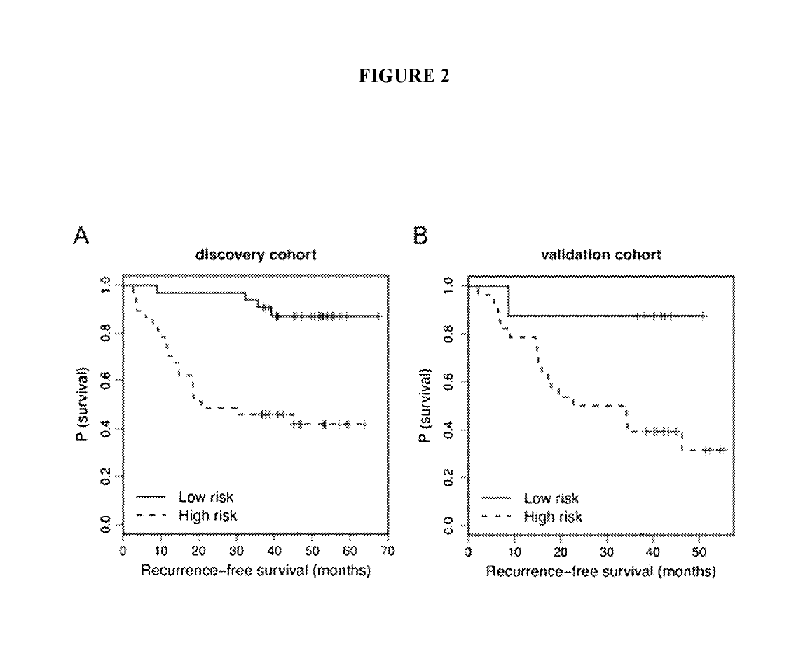

The performance of the 5 miRNAs identified by Cox analysis was then evaluated by logistic regression analysis. In the discovery cohort, the addition of the 5-miRNA signature to a base risk model containing stage as a predictor improved the performance of the classifier, demonstrated by an increase in the area under the ROC curve (AUC) from AUC=0.77 (95% CI, 0.66-0.89) of stage alone to AUC=0.84 (95% CI, 0.73-0.94) with the 5-miRNA signature. Using a cutoff to separate the discovery cohort into high and low recurrence risk groups (sensitivity=84%, specificity=64%), the two groups had significant separation of RFS (p<0.0001, FIG. 2A). The model was applied to predict recurrence in the validation cohort, and the same cutoff was used to define high and low recurrence risk groups (sensitivity=95%, specificity=41%). Performance of the classifier was maintained, with significant separation of RFS (p=0.033, FIG. 2B).

The second and third logistic models identified using the discovery cohort used a set of 6 miRNAs (miR-103, -15b, -23b, -30d, -423-5p, -425) and 5 miRNAs (miR-222, -23a, -26a, -339-3p, -423.5p), respectively, to distinguish recurred from non-recurred patients. Adjusting for thickness, both models achieve an AUC above 80% when used to classify the discovery cohort. Using a cut-off value to classify the discovery cohort into high and low recurrence risk groups, the resulting groups had significantly different RFS distributions (log-rank test, p-values <0.005, data not shown). In the validation cohort, logistic models using the second and third sets of miRNAs with adjustment for thickness yielded ROCs with AUCs above 90%. A cut-off value for each model was used to classify the validation cohort into high and low risk groups, and Kaplan Meier curves for RFS showed significant differences between the two groups (log-rank test, p-values <0.005, data not shown). Cut-off values were selected to maximize sensitivity (>80%) and maintain specificity of about 70%. Slight variations of the cut-off points yielded similar separation between the Kaplan-Meier curves and similar p-values for log-rank tests. This demonstrates risk models that incorporate selected miRNAs with adjustment for primary tumor stage and thickness can discriminate patients with long versus short RFS.

A subset of the second model of miRNAs included a set of 5 miRNAs (miR-30d, -15b, -23b, -423-5p, -425) (denoted Subset Model 2) to distinguish recurred from non-recurred patients. Adjusting for stage and thickness, the model was significant (p<0.0001), all miRNAs were significant predictors of recurrence and the risk model achieved an AUC=0.89 when used to classify cohort 1. Using an optimal cut-off value to classify the patients into high and low recurrence risk groups (sensitivity=88%, specificity=75%), the resulting groups again had clear separation in survival probability. Because of patient heterogeneity, we used two logistic models to identify a total of 9 miRNAs from the subset models (miR-150, -15b, -199a-5p, -33a, -424, -30d, -23b, -423-5p and -425) that showed promise as biomarkers of recurrence at initial diagnosis. Though some did not reach statistical significance in the models, none of the identified miRNAs have been previously studied in the sera of melanoma patients. Therefore, all were retained for further evaluation to avoid eliminating potentially useful miRNAs.

In Kaplan Meier curves comparing RFS (recurrence free survival) defined by 3 miRNA-based signatures, RFS distributions are significantly different by log rank test for high and low recurrence risk groups defined by miRNA-based model 1 (tumor thickness+7 miRNAs) (miR-150, miR-15b, miR-199a-5p, miR-33a, miR-423-5p, miR-424 and miR-let-7d) in the discovery cohort and the independent cohort; miRNA-based model 2 (tumor thickness+6 miRNAs) (miR-103, miR-15b, miR-23b, miR-30d, miR-423-5p and miR-425) in the discovery cohort and the independent cohort; and miRNA-based model 3 (tumor thickness+5 miRNAs) (miR-222, miR-23a, miR-26a, miR-339-3p, miR-423.5p) in the discovery cohort and the independent cohort (data not shown).

A Signature of Candidate miRNAs as Predictors of Recurrence in Stage II Melanoma

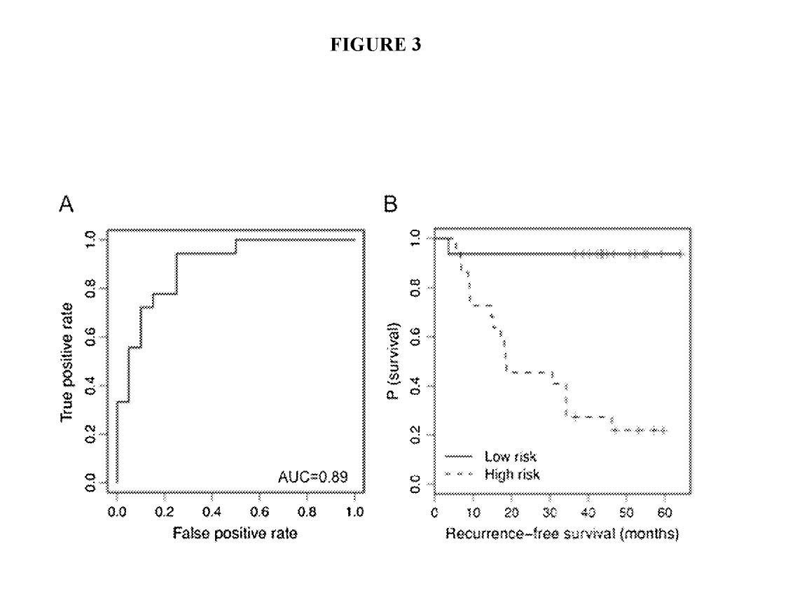

Recurrence status among all stage II patients was relatively balanced, with 19 recurred and 20 non-recurred after ≧3 years follow up. Thus, this group was selected for subgroup analyses as proof-of-principle that serum-based miRNAs have prognostic potential beyond stage. Stage II patients pose a particularly difficult clinical challenge. Though still localized melanoma, Stage II patients have significantly lower survival rates than stage I patients, with 5-year survival rates from 53% to 82% compared to >90%, respectively [33]. In our cohort of stage II patients, a logistic risk model with thickness alone was able to distinguish recurred from non-recurred patients with an AUC=0.75 (p=0.06). With the addition of 3 of the miRNAs prioritized for further evaluation (miR-423-5p, -424, -199a-5p), the performance of the classifier improved (AUC=0.89, FIG. 3A), and 2 of the miRNAs were significant predictors of recurrence in the model (miR-423-5p: Odds Ratio (OR), 0.038; 95% CI, 0.0031-0.46; p=0.008; miR-424: OR, 3.64; 95% CI, 1.19-11.1; p=0.018). Four-fold cross validation demonstrated an AUC=0.81. Using Youden's index of ROC curve as a cutoff to separate patients into high and low recurrence risk groups (sensitivity=94%, specificity=75%), the two groups had significant separation of RFS (p<0.0001, FIG. 3B).

Evaluation of recurrence risk models containing miRNA signatures

To evaluate the utility of the Subset models, each was fitted in cohort 2. Subset Model 1 classified recurred from non-recurred with an AUC=0.98, and when using a cut-point with sensitivity=95% and specificity=88%, resulted in clear separation of RFS of high and low recurrence risk groups. Subset Model 2 yielded an ROC with AUC=0.96 and also resulted in clear separation of RFS of predicted high and low recurrence risk groups using a cut point with sensitivity=95% and specificity=94%.

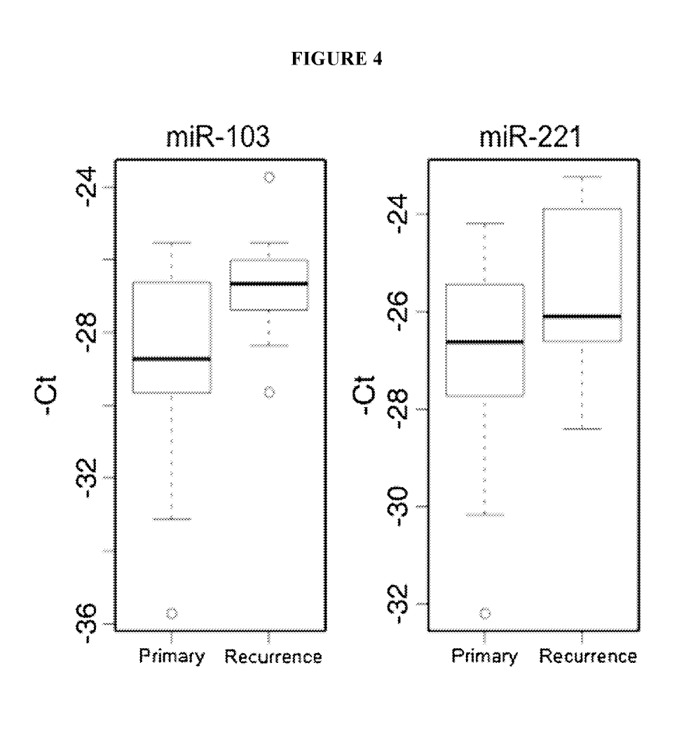

To evaluate the clinical feasibility of using circulating miRNAs in post-resection surveillance for recurrence, we first measured the expression of 7 miRNAs, selected from our prioritized panel of 11 miRNAs based on previously supported roles in cancer progression and/or diagnostic utility and from tissue-based miRNA studies supporting an association with melanoma progression[27, 28], in 10 matched serum samples collected pre- and postresection of primary melanoma. Mean expression levels of all 7 miRNAs were reduced after operation, but only the difference in mean expression of miR-221, -222 and −423-5p reached statistical significance (p<0.05). Next, we measured the expression of these miRNAs in pre- and post-recurrence serum samples from a subset of the discovery cohort (n=17). Serum levels of all 7 miRNAs were increased, with mean expression of 2 miRNAs (miR-103 and -221) showing statistically significant post-recurrence elevation (p<0.025, FIG. 4).

### DISCUSSION

We present the first study demonstrating the potential prognostic value of serum miRNAs in melanoma. Specifically, we provide evidence that serum miRNAs can serve as biomarkers for improved identification of high risk melanoma patients at the time of primary diagnosis. By doing so, we introduce biological subclassifiers that could add to the utility of stage and could help account for the latent heterogeneity in melanoma. This is increasingly important as better adjuvant therapies are developed, since it would enable clinicians to target those likely to recur towards aggressive treatment.

We identify miRNAs that are potentially markers of disease progression in melanoma patients. In other malignancies, such as colon, liver, pancreatic, prostate, and ovarian cancers, serum tumor markers have become part of the standard of care in surveillance for recurrence, at times used in lieu of periodic imaging in asymptomatic patients (34). In melanoma, however, there are no widely available circulating markers that can facilitate early detection of relapse. Here, we show support for an association between serum miRNAs and tumor burden, a feature that if further developed, could be exploited by incorporation of serum miRNAs assays in routine surveillance for melanoma recurrence.

While there are several tissue-based and in vitro studies of miRNA dysregulation in melanoma [28, 32, 35-39], little is known about the “source” of serum miRNAs [19,21]. There is speculation that the source is not limited to the tumor and can include miRNAs shed from the tumor microenvironment or from circulating immune cells that also reflect the myriad of events involved in the initiation and development of malignancy. This suggests that, though tissue-based analyses are useful for understanding cancer biology, there is a complimentary role for studying circulating miRNAs as an independent source of biologically and clinically relevant information.

Growing interest in circulating miRNAs as biomarkers has resulted in several studies exploring their potential in solid cancers. To date, the emphasis has been placed on elucidating the diagnostic capabilities of blood-based miRNAs (16, 17), with only two studies in melanoma investigating the ability of circulating miRNAs to distinguish melanoma patients from controls (40, 35). In the first, the authors studied serum miR-221 levels and demonstrated a correlation with tumor thickness (40). The second used array screening to identify unique miRNA expression profiles in peripheral blood cells of melanoma patients compared to healthy controls (35), which requires analysis of fresh blood specimens, and limits the practicality of the approach.

While we are the first to identify the prognostic relevance in melanoma of three of the miRNAs included in the recurrence risk signature (miR-199a-5p, -33a, -424), the biological function of all the identified miRNAs have begun to be elucidated in tissue and in vitro studies. miR-150 directly targets MUC4 in pancreatic cancer cells, an aberrantly overexpressed transmembrane mucin promoting growth, invasion and metastasis. miR-150 overexpression inhibits growth, clonogenicity, migration and invasion, and enhances intracellular adhesion in pancreatic cancer cells [41]. Clinically, miR-150 was elevated in melanoma tissues of patients with longer post-recurrence survival[39], also supporting a tumor suppressor role. Conflicting data exist regarding a pro-proliferative effect of ectopic expression of miR-150 in gastric cancer both in vitro and in vivo, at least in part due to repression of the tumor suppressor EGR2 resulting in translational arrest [42]. Our findings are in line with an oncogenic role for miR-150, with higher circulating expression of miR-150 in patients with a high recurrence risk. However, our findings can also be due to the possibility that serum miRNAs reflect the systemic immune response as miR-150 has a role in the modulation of the T-cell development. Specifically, via modulation of NOTCH3, overexpression of miR-150 has adverse effects on T-cell proliferation and survival, resulting in decreased antitumor immunity and subsequent progression[43].

In our model, patients with high recurrence risk had lower levels of miR-15b, similar to findings in a recent study showing an association between reduced miR-15b expression, chemotherapeutic resistance and poor prognosis in patients with tongue squamous cell carcinoma [44]. The same study identified BIM1 as a functional target of miR-15b, through which BIM1 overexpression due to reduction of miR-15b regulates epithelial to mesenchymal transition and chemoresistence. Also in line for a tumor suppressor role for miR-15b is its direct regulation of the critical anti-apoptotic Bcl-2 protein [45]. On the other hand, a previous study in melanoma demonstrated that miR-15b downregulation in miR-15b high melanoma cell lines resulted in decreased proliferation and increased apoptosis and clinically, found increased tissue-based expression of miR-15b in melanoma FFPE samples from patients with shorter recurrence-free and overall survival[31]. However, these differences may be attributable to host-specific effects reflected by measurement of circulating miRNAs as opposed to solely tumor-specific effects.

While we are the first to demonstrate the prognostic potential of miR-199a-5p in melanoma, with higher expression in high recurrence risk patients, its diagnostic potential was supported by a study that found miR-199a-5p overexpression in blood cells of melanoma patients compared to healthy controls [35]. Our data, taken with a recent study that found miR-199a-5p was one of several miRNAs overexpressed in a small sample of brain-metastatic compared to primary colorectal carcinomas [46], invites speculation that miR-199a-5p has a role in the process of dissemination beyond its role in carcinogenesis, which is in part via modulation of Brm-type SWI/SNF activity [47]. Future studies will be needed to further define the role of this miRNA in cancer metastasis, and specifically in melanoma.

Strong evidence exists supporting a tumor suppressor function for miR-33a via repression of proto-oncogene Pim-1 [48] and inhibition of expression of cyclin-dependent kinase 6 (CDK6) and cyclin D1 (CCDN1) [49], both of which results in reduced cellular proliferation and cell-cycle progression. Though not previously studied in melanoma, our data is also suggestive of a tumor suppressive function for miR-33a, with lower levels of miR-33a associated with high recurrence risk scores. As with miR-33a, the prognostic or functional relevance of miR-424 has not been studied in melanoma, but roles have been previously defined for miR-424 in HIF-1a/HIF-2a mediated angiogenesis [50] and regulation of monocyte/macrophage differentiation[51]. Given these roles of miR-424 in tumor formation and dissemination (i.e. angiogenesis) and in systemic immune responses, it is not surprising that, in measuring circulating miRNAs, we found patients with high recurrence risk to have elevated levels of miR-424 in the recurrence risk model. Though the biological role and mechanistic relationship of each of these components are not completely understood, particularly as they relate to melanoma, these miRNA prognostic classifiers can still be clinically useful [52].

The significant post-operative reduction and subsequent elevation at recurrence of the miRNAs identified as having potential in detection of disease relapse (miR-103, -221, -222, -423-5p) is consistent with existing data delineating their roles in cancer progression. miR-103, via downregulation of the enzyme Dicer, promotes cell migration and invasion in breast cancer cells in vitro [24]. Clinically, high levels of miR-103 are associated with metastasis and poor outcome in breast cancer patients [24]. In vitro and in vivo studies support oncogenic roles for miR-221/222, which function to increase invasion and migration capabilities as well as proliferative growth rate in melanoma by targeting of c-kit, p27, and p57 [28]. Though the functional relevance and target genes of miR-423-5p have yet to be uncovered, serum levels were significantly higher in gastric cancer patients compared to healthy controls and it was identified as part of 5-miRNA signature for gastric cancer diagnosis [26]. Together with our results that demonstrate higher levels at the time of recurrence, we can begin to speculate about a possible oncogenic role for miR-432-5p.

We acknowledge that our study has certain limitations. First, we performed unbiased serum miRNA expression profiling using a system demonstrated to have the highest sensitivity for analyzing serum miRNAs [23]. With 1,500+ transcribed miRNAs identified in the human genome [16], the use of a panel containing 355 miRNA assays could potentially be limiting. However, the miRNAs included were identified as being a comprehensive set of miRNAs expressed in serum from extensive genome wide qPCR screening of normal and diseased samples, including melanoma patients [23]. Next, given the expected imbalance in recurrence status among stage I and III patients, we cannot speculate about a predictive model that would work across all stages. However, we are able to prioritize a manageable panel of miRNAs for future testing that show potential as circulating biomarkers with clinical relevance in identifying primary melanoma patients at high risk for recurrence.

The evolving paradigm shift towards a molecular characterization of melanoma to improve prognostic accuracy, detect recurrence, and better guide management decisions has largely been devoid of blood-based miRNA studies. We show that serum-based miRNAs demonstrate hallmarks of useful tumor markers, namely easy detection in accessible samples and promising clinical utility and applicability in melanoma. We demonstrate support for the use of serum miRNAs as clinically useful non-invasive biomarkers that could potentially enhance the utility of current prognostic factors in predictive models of melanoma recurrence. This would allow for improved stratification of a heterogeneous cancer that could be influential in clinical decision-making. Specifically, the development of serum miRNAs into clinical assays can refine criteria for more extensive staging procedures, such as sentinel lymph node biopsy, and for inclusion in clinical trials. Moreover, the potential exists for serum miRNAs to not only guide follow-up recommendations and allow for better allocation of resources, but also to improve the power of the current arsenal of imaging and laboratory tests used in routine surveillance. Future large prospective studies focusing on the identified serum-based miRNAs stand to have a potentially large clinical impact in aiding the early identification of primary melanoma patients with high risk for recurrence and in the timely detection of disease relapse.

### REFERENCES

1. Howlader N, Noone A M, Krapcho M, et al. SEER Cancer Statistics Review, 1975-2008 [Internet]. Available from URL: seer.cancer.gov/csr/1975_{—}2008/[accessed Dec. 8, 2011].
2. Tsao H, Atkins M B, Sober A J. Management of cutaneous melanoma. N Engl J Med 2004; 351(10): 998-1012.
3. Garbe C. A rational approach to the follow-up of melanoma patients. Recent Results Cancer Res 2002; 160: 205-15.
4. Balch C M, Buzaid A C, Soong S J, et al. Final version of the American Joint Committee on Cancer staging system for cutaneous melanoma. J Clin Oncol 2001; 19(16): 3635-48.
5. Leiter U, Buettner P G, Eigentler T K, Forschner A, Meier F, Garbe C. Is detection of melanoma metastasis during surveillance in an early phase of development associated with a survival benefit? Melanoma Res 2010; 20(3): 240-6.
6. Grange F, Vitry F, Granel-Brocard F, et al. Variations in management of stage I to stage III cutaneous melanoma: a population-based study of clinical practices in France. Arch Dermatol 2008; 144(5): 629-36.
7. Hofmann U, Szedlak M, Rittgen W, Jung E G, Schadendorf D. Primary staging and follow-up in melanoma patients—monocenter evaluation of methods, costs and patient survival. Br J Cancer 2002; 87(2): 151-7.
8. Bast R C, Jr., Ravdin P, Hayes D F, et al. 2000 update of recommendations for the use of tumor markers in breast and colorectal cancer: clinical practice guidelines of the American Society of Clinical Oncology. J Clin Oncol 2001; 19(6): 1865-78.
9. Berger A J, Camp R L, Divito K A, Kluger H M, Halaban R, Rimm D L. Automated quantitative analysis of HDM2 expression in malignant melanoma shows association with early-stage disease and improved outcome. Cancer Res 2004; 64(23): 8767-72.
10. Deichmann M, Benner A, Bock M, et al. S100-Beta, melanoma-inhibiting activity, and lactate dehydrogenase discriminate progressive from nonprogressive American Joint Committee on Cancer stage IV melanoma. J Clin Oncol 1999; 17(6): 1891-6.
11. Tarhini A A, Stuckert J, Lee S, Sander C, Kirkwood J M. Prognostic significance of serum S100B protein in high-risk surgically resected melanoma patients participating in Intergroup Trial ECOG 1694. J Clin Oncol 2009; 27(1): 38-44.
12. Garbe C, Paul A, Kohler-Spath H, et al. Prospective evaluation of a follow-up schedule in cutaneous melanoma patients: recommendations for an effective follow-up strategy. J Clin Oncol 2003; 21(3): 520-9.
13. Bartel D P. MicroRNAs: genomics, biogenesis, mechanism, and function. Cell 2004; 116(2): 281-97.
14. Lu J, Getz G, Miska E A, et al. MicroRNA expression profiles classify human cancers. Nature 2005; 435(7043): 834-8.
15. Mitchell P S, Parkin R K, Kroh E M, et al. Circulating microRNAs as stable blood-based markers for cancer detection. Proc Natl Acad Sci USA 2008; 105(30): 10513-8.
16. Cortez M A, Bueso-Ramos C, Ferdin J, Lopez-Berestein G, Sood A K, Calin G A. MicroRNAs in body fluids—the mix of hormones and biomarkers. Nat Rev Clin Oncol 2011.
17. Cortez M A, Calin G A. MicroRNA identification in plasma and serum: a new tool to diagnose and monitor diseases. Expert Opin Biol Ther 2009; 9(6): 703-11.
18. Lawrie C H, Gal S, Dunlop H I M, et al. Detection of elevated levels of tumour-associated microRNAs in serum of patients with diffuse large B-cell lymphoma. Br J Haemato! 2008; 141(5): 672-5.
19. Cheng H, Zhang L, Cogdell D E, et al. Circulating plasma MiR-141 is a novel biomarker for metastatic colon cancer and predicts poor prognosis. PLoS One 2011; 6(3): e17745.
20. Hu Z, Chen X, Zhao Y, et al. Serum microRNA signatures identified in a genome-wide serum microRNA expression profiling predict survival of non-small-cell lung cancer. J Clin Oncol 2010; 28(10): 1721-6.
21. Boeri M, Verri C, Conte D, et al. MicroRNA signatures in tissues and plasma predict development and prognosis of computed tomography detected lung cancer. Proc Natl Acad Sci USA 2011; 108(9): 3713-8.
22. Wich L G, Hamilton H K, Shapiro R L, et al. Developing a multidisciplinary prospective melanoma biospecimen repository to advance translational research. Am J Transl Res 2009; 1(1): 35-43.
23. Segura M F, Hanniford D, Menendez S, et al. Aberrant miR-182 expression promotes melanoma metastasis by repressing FOXO3 and microphthalmia-associated transcription factor. Proc Natl Acad Sci USA 2009; 106(6): 1814-9.
24. Felicetti F, Errico M C, Segnalini P, Mattia G, Care A. MicroRNA-221 and -222 pathway controls melanoma progression. Expert Rev Anticancer Ther 2008; 8(11): 1759-65.
25. Poo-Hwu W J, Ariyan S, Lamb L, et al. Follow-up recommendations for patients with American Joint Committee on Cancer Stages I-III malignant melanoma. Cancer 1999; 86(11): 2252-8.
26. Wang Y, Chen H, Li R, Duan N, Lewis-Fernandez R. Prediction-Based Structured Variable Selection through the Receiver Operating Characteristic Curves. Biometrics 2011; 67(3): 896-905.
27. Balch C M, Gershenwald J E, Soong S J, et al. Final version of 2009 AJCC melanoma staging and classification. J Clin Oncol 2009; 27(36): 6199-206.
28. Kanemaru H, Fukushima S, Yamashita J, et al. The circulating microRNA-221 level in patients with malignant melanoma as a new tumor marker. J Dermatol Sci 2011; 61(3): 187-93.
29. Leidinger P, Keller A, Borries A, et al. High-throughput miRNA profiling of human melanoma blood samples. BMC Cancer 2010; 10: 262.
30. Mueller D W, Rehli M, Bosserhoff A K. miRNA expression profiling in melanocytes and melanoma cell lines reveals miRNAs associated with formation and progression of malignant melanoma. J Invest Dermatol 2009; 129(7): 1740-51.
31. Caramuta S, Egyhazi S, Rodolfo M, et al. MicroRNA expression profiles associated with mutational status and survival in malignant melanoma. J Invest Dermatol 2010; 130(8): 2062-70.
32. Philippidou D, Schmitt M, Moser D, et al. Signatures of microRNAs and selected microRNA target genes in human melanoma. Cancer Res 2010; 70(10): 4163-73.
33. Segura M F, Belitskaya-Levy I, Rose A E, et al. Melanoma MicroRNA signature predicts post-recurrence survival. Clin Cancer Res 2010; 16(5): 1577-86.
34. Satzger I, Mattern A, Kuettler U, et al. MicroRNA-15b represents an independent prognostic parameter and is correlated with tumor cell proliferation and apoptosis in malignant melanoma. Int J Cancer 2010; 126(11): 2553-62.
35. Gaziel-Sovran A, Segura M F, Di Micco R, et al. miR-30b/30d Regulation of Ga1NAc Transferases Enhances Invasion and Immunosuppression during Metastasis. Cancer Cell 2011; 20(1): 104-18.
36. Simon R. Roadmap for developing and validating therapeutically relevant genomic classifiers. J Clin Oncol 2005; 23(29): 7332-41.
37. Jensen S G, Lamy P, Rasmussen M E I, et al. Evaluation of two commercial global miRNA expression profiling platforms for detection of less abundant miRNAs. BMC Genomics 2011; 12(1): 435.

### Example 2

### A Serum-Based miRNA Signature Predicts Recurrence in Primary Melanoma Patients

Identification of primary melanoma patients at the highest risk of recurrence remains a critical challenge. The above study and Example provides an array-based identification of a set of serum miRNAs as predictors of recurrence in melanoma patients at the time of primary diagnosis. In this study, we refined the miRNA signature in a cohort of patients balanced by recurrence status and included normalizer miRNAs. Twelve miRNAs were tested for inclusion in the recurrence risk signature, while 5 miRNAs were tested as potential normalizers/internal controls for the assay and 1 miRNA was included for quality control purposes.

Expression levels of 18 miRNAs (including 6 potential normalizers) were measured using qRT-PCR (Exiqon) in serum prospectively collected at diagnosis of 201 patients (median FU of survivors 75.7 months). The miRNAs measured were as follows:

miR-15b/miR-15b-5p

miR-23b/miR-23b-3p

miR-30/miR-30d-5p

miR-33a/miR-33a-5p

miR-103/miR-103a-3p

miR-150/miR-150-5p

miR-199a-5p

miR-423-5p

miR-424/miR-424-5p

miR-425/miR-425-5p

miR-let-7d/miR-let-7d-5p

miR-221/miR221-3p

miR-142-3p (normalizer)

miR-451/miR-451a (normalizer)

miR-30c/miR-30c-5p (normalizer)

miR-181a/miR-181a-5p (normalizer)

miR-27b/miR-27b-3p (normalizer)

miR-23a/miR-23a-3p (quality control)

MiRNAs were ranked by univariate association with recurrence. Top-ranking miRNAs were candidates for the multivariate logistic regression model. The miRNA signature was selected by minimizing Akaike's information criterion. The selected miRNA signature was evaluated by identifying the area under the ROC curve in the training cohort and an independent validation cohort of 82 patients (median FU of survivors 39.0 months).

A predictive signature of four miRNAs significantly separated recurrence-free and overall survival in both the training and validation cohorts (training RFS and OS P<0.001, validation RFS P<0.001, OS P=0.002). The four miRNAs providing a best predictive signature were: miR-425-5p, miR-150-5p, miR-23b-3p and miR-15b-5p. The best normalization was achieved using two miRNAs as internal controls: miR-30c-5p and miR-181a-5p.

The model improved prediction of recurrence over stage alone, increasing AUC from 0.69 to 0.75 in the training cohort and from 0.76 to 0.78 in the validation cohort. Notably, the model performed well in predicting recurrence of stage I patients in the validation cohort (AUC=0.88), but performed poorly in classifying stage II and III patients.

In summary, our serum-based miRNA recurrence risk signature improved the classification of primary melanoma patients over stage alone. Our data suggest that serum-based miRNAs have prognostic potential beyond stage, especially for early stage patients.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein. The summary, description, materials and methods and drawings are, accordingly, to be regarded in an illustrative rather than restrictive sense.

Various references, including patents and printed publications, are cited throughout this Specification, each of which is incorporated herein by reference in its entirety.

1. A method for detecting or evaluating melanoma in a mammal comprising:
(a) obtaining a sample of blood or serum from said mammal;
(b) measuring the expression or activity of two or more miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p; and
(c) comparing the expression or activity of said two or more miRNAs to that in or from a reference sample or to the expression or activity of one or more normalization or control miRNA;
wherein the expression or activity of said two or more miRNAs is altered relative to the reference sample or the normalization or control miRNA.

2. The method of claim 1 wherein the expression or activity of five or more miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p are measured.

3. The method of claim 1 wherein the expression or activity of a set of miRNAs is measured, the set selected from:
(a) a set comprising miRNAs miR-150, miR-15b, miR-199-5p, miR-33a, miR-423-5p, miR-424, and miR-let-7d;
(b) a set comprising miRNAs miR-103, miR-15b, miR-23b, miR-30d, miR-423-5p and miR-425;
(c) a set comprising miRNAs miR-222, miR-23a, miR-26a, miR-339-3p and miR-423.5p;
(d) a set comprising miRNAs miR-30d, miR-1991-5p, miR-22, miR-423.5p and miR-424;
(a) a set comprising miRNAs miR-150, miR-15b, miR-199-5p, miR-33a, and miR-424;
(b) a set comprising miRNAs miR-423-5p, miR-424, and miR-199a-5p;
(c) a set comprising miRNAs miR-103, mir-221, miR-222, and miR-423-5p; and
(d) a set comprising miRNAs miR-150, miR-425, miR-15b and miR-23b.

4. The method of claim 1 wherein altered expression or activity of at least two or more miRNAs indicates an increased risk of recurrence in said mammal.

5. A method for evaluating the risk of recurrence in a subject with melanoma comprising:
(a) obtaining a serum sample from said subject;
(b) measuring the expression or activity of at least two miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p in said sample; and
(c) optionally comparing the expression or activity of said at least two miRNAs to that in or from at least one reference sample or set, or to the expression or activity of at least one normalization or control miRNA;
wherein, if the expression or activity of at least two of said miRNAs is altered relative to the reference sample or set or relative to the normalization or control miRNA, the subject is at risk of recurrence.

6. The method of claim 5 wherein the expression or activity of five or more miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p are measured.

7. The method of claim 5 wherein the expression or activity of miRNAs miR-150, miR-15b, miR-23b and miR-425 are measured.

8. The method of claim 1 or 5 wherein the normalization or control miRNA is one or more miRNA selected from miR-142-3p, miR-451, miR-30c, miR-181a, miR-27b and miR-23a.

9. The method of claim 5 wherein the expression or activity of a set of miRNAs is measured, the set selected from:
(a) a set comprising miRNAs miR-150, miR-15b, miR-199-5p, miR-33a, miR-423-5p, miR-424, and miR-let-7d;
(b) a set comprising miRNAs miR-103, miR-15b, miR-23b, miR-30d, miR-423-5p and miR-425;
(c) a set comprising miRNAs miR-222, miR-23a, miR-26a, miR-339-3p and miR-423.5p; and
(d) a set comprising miRNAs miR-30d, miR-199a-5p, miR-22, miR-423.5p and miR-424;
(e) a set comprising miRNAs miR-150, miR-15b, miR-199-5p, miR-33a, and miR-424;
(f) a set comprising miRNAs miR-423-5p, miR-424, and miR-199a-5p;
(g) a set comprising miRNAs miR-103, mir-221, miR-222, and miR-423-5p; and
(h) a set comprising miRNAs miR-150, miR-425, miR-15b and miR-23b.

10. The method of claim 5 wherein the subject is a subject with Stage I, Stage II or Stage III melanoma.

11. The method of claim 9 wherein the expression or activity of a set of miRNAs is measured, the set comprising miRNAs miR-30d, miR-199a-5p, miR-222, miR-423.5p and miR-424.

12. The method of claim 9 wherein the expression or activity of a set of miRNAs is measured, the set comprising miRNAs miR-425, miR-150, miR-15b and miR-23b.

13. The method of claim 12 wherein the subject is a subject with Stage I melanoma.

14. A kit for prognosticating melanoma a cancer comprising a composition capable of binding to a portion of at least two microRNAs, wherein the microRNAs are selected from the group consisting of miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p, wherein altered expression levels of the microRNAs in a serum sample is prognostic for the cancer, or prognosticates the cancer.

15. (canceled)

16. The kit of claim 14 comprising a composition capable of binding to a portion of at least two microRNAs, wherein the microRNAs are selected from:
(a) a set comprising miRNAs miR-150, miR-15b, miR-199-5p, miR-33a, miR-423-5p, miR-424, and miR-let-7d;
(b) a set comprising miRNAs miR-103, miR-15b, miR-23b, miR-30d, miR-423-5p and miR-425;
(c) a set comprising miRNAs miR-222, miR-23a, miR-26a, miR-339-3p and miR-423.5p;
(d) a set comprising miRNAs miR-30d, miR-199a-5p, miR-222, miR-423.5p and miR-424; and
(e) a set comprising miRNAs miR-150, miR-425, miR-15b and miR-23b.

17. A composition which comprises at least two nucleic acids, antagomirs or oligonucleotides complementary to at least two miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p.

18. The composition of claim 17 wherein the nucleic acids, antagomirs or oligonucleotides are at least about 80% identical to at least two miRNAs selected from miR-150, miR-15b, miR-199a-5p, miR-33a, miR-423-5p, miR-424, miR-let-7d, miR-103, miR-23b, miR-30d, miR-425, miR-222, miR-23a, miR-26a and miR-339-3p.

19. The composition of claim 17 wherein the microRNAs are selected from:
(a) a set comprising miRNAs miR-150, miR-15b, miR-199-5p, miR-33a, miR-423 5p, miR-424, and miR-let-7d;
(b) a set comprising miRNAs miR-103, miR-15b, miR-23b, miR-30d, miR-423-5p and miR-425;
(c) a set comprising miRNAs miR-222, miR-23a, miR-26a, miR-339-3p and miR-423.5p;
(d) a set comprising miRNAs miR-30d, miR-199a-5p, miR-222, miR-423.5p and miR-424; and
(e) a set comprising miRNAs miR-150, miR-425, miR-15b and miR-23b.

20. The method of claim 5 wherein the normalization or control miRNA is one or more miRNA selected from miR-142-3p, miR-451, miR-30c, miR-181a, miR-27b and miR-23a.
